(19) United States
(12) Patent Application Publication
(10) Pub. No.: US 2025/0276178 A1
(43) Pub. Date: Sep. 4, 2025

(54) SYSTEM AND METHOD FOR TRANSVASCULAR THERAPY

(71) Applicants: Kalyanam SHIVKUMAR, Los Angeles, CA (US); Michael DACEY, Los Angeles, CA (US); Jeffrey ARDELL, Los Angeles, CA (US); Anthony Chin-Hsui WANG, Los Angeles, CA (US); Wi Jin KIM, Los Angeles, CA (US); Anthony WANG, Los Angeles, CA (US); THE REGENTS OF THE UNIVERSITY OF CALIFORNIA, Oakland, CA (US)

(72) Inventors: Geoffrey P. COLBY, Los Angeles, CA (US); Michael DACEY, Los Angeles, CA (US); Jeffrey ARDELL, Los Angeles, CA (US); Kalyanam SHIVKUMAR, Los Angeles, CA (US); Anthony Chin-Hsui WANG, Los Angeles, CA (US); Wi Jin KIM, Los Angeles, CA (US)

(21) Appl. No.: 18/857,727

(22) PCT Filed: Apr. 18, 2023
(86) PCT No.: PCT/US2023/065908

§ 371 Date: Oct. 17, 2024

Related U.S. Application Data
(60) Provisional application No. 63/332,119, filed on Apr. 18, 2022.

(51) Int. Cl. A61N 1/36; A61N 1/05

(52) U.S. Cl. CPC A61N 1/3606; A61N 1/0551

(57) ABSTRACT

The present disclosure includes systems and methods for transvascular neuromodulation. Some aspects are directed to a system and method for applying transvascular neuromodulation to a nervous system tissue for treating neurological disorders. In one aspect, a method for stimulating nervous system tissue using an endovascular transvascular technique includes: inserting a therapy delivery device comprising an infusion device into a vessel of a patient; positioning the therapy delivery device in the vessel near the nervous system tissue; and applying, from the infusion device, a therapeutic across a wall of the vessel into tissue of the patient to treat the nervous system tissue and a neurologic condition.

This application claims priority of U.S. Provisional Patent Application No. 63/332,119, filed Apr. 18, 2022, which is hereby incorporated by reference in its entirety.

This invention was made with government support under EB025138 awarded by the National Institutes of Health. The government has certain rights in the invention.

### TECHNICAL FIELD

Aspects of the present disclosure relate generally to apparatus, systems, and methods for endovascular transvascular therapy and, more particularly but without limitation, to a system for applying transvascular neuromodulation to nervous system tissue and a method of use therefore in treating cerebral vasospasm.

### BACKGROUND

Neuromodulation involves a therapeutic approach applied to the central nervous system of a body to treat various human disorders. Various therapeutic approaches may be used, and generally the approach corresponds to the human disorder to be treated. These include therapeutic approaches electrical and chemical treatments. For example, neuromodulation can involve electrical stimulation, chemical lesioning, stimulation/modulation as well as gene therapy and administration of stem cells. The neuromodulation therapy may be applied to any portion or portions of the central nervous system, including the brain, cranial nerves, spinal cord and all associated nerves and neural structures in the human body.

Electrical stimulation of neural tissue is becoming an increasingly preferred form of therapy for certain neurological conditions and disorders where existing therapies generate intolerable side effects, require repeated administration of treatment, or are simply ineffective in a subset of patients. Electrical stimulation provides distinct advantages over surgical lesioning techniques since electrical stimulation is a reversible and adjustable procedure that provides continuous benefits as the patient's disease progresses and the patient's symptoms evolve. Currently, electrical stimulation of peripheral nerves and the spinal cord is approved for treatment of neuropathic pain. With respect to deep brain targets, electrical stimulation of the subthalamic nucleus and the globus pallidus interna is approved for treatment of Parkinson's disease and electrical stimulation of the Ventral intermediate nucleus is approved for treatment of essential tremor. There remains a need for further forms of neuromodulation to treat these and other disorders.

Endovascular surgery and the corresponding approaches involve minimally invasive surgery that utilize small incisions and catheters or other instruments which are inserted into and guided through blood vessels. A transvascular approach is a particular type of endovascular approach in which a catheter is advanced through vessels as in a standard endovascular approach and then the catheter is used to deliver therapies through vessel walls directly to tissue or organs deep inside the body. This technique keeps an injected agent at high concentration near the target and helps it from spreading into the rest of the body, and enables non-systemic delivery of therapeutic agents directly across any blood vessel.

Cerebral vasospasm is a disease of pathologic narrowing of the cerebral vasculature leading to diminished cerebral blood flow, resulting in significant morbidity and stroke. To ensure adequate brain perfusion, multiple mechanisms tightly regulate cerebral blood flow through myogenic, neurogenic, metabolic and endothelial processes known as autoregulation. Cerebral vasospasm is a disease of impaired autoregulation, resulting in potentially reversible narrowing of blood vessels, often leading to permanent stroke and poor patient outcomes. It occurs in up to 50-90% of patients with aneurysmal subarachnoid hemorrhage, and 60% of patients with traumatic brain injury, making it one of the leading causes of preventable morbidity in these patients. Yet, there are limited durable treatment modalities for patients experiencing cerebral vasospasm; intra-arterial calcium channel blockers are moderately effective but short-acting, and balloon angioplasty has limited access to distal vessels with higher risks of vessel dissection and rupture. Hence, targeted therapies to augment cerebral blood flow in disease states such as cerebral vasospasm are of immediate clinical importance.

Although the exact mechanism of cerebral vasospasm remains unclear, sympathetic hyperactivity has been shown to be a significant contributor. In the art, there is a need to better understand and define the role of sympathetic-mediated vasospasm, but to date, a robust model does not exist, limiting mechanistic understanding and therapeutic development.

### SUMMARY

The present disclosure describes systems, devices, and methods for treating cerebral vasospasms. For example, the present disclosure describes.

In one aspect, a method for stimulating nervous system tissue using an endovascular transvascular technique includes: inserting a therapy delivery device comprising an infusion device into a vessel of a patient; positioning the therapy delivery device in the vessel near the nervous system tissue; and applying, from the infusion device, a therapeutic across a wall of the vessel into the nervous system tissue of the patient to treat the nervous system tissue and a neurologic condition.

In another aspect, a method for applying therapy using an endovascular transvascular technique includes: receiving, by a therapy delivery device while in a vessel of a patient, a therapeutic, the therapy delivery device comprising an infusion device; and providing, by the infusion device, the therapeutic medication across a wall of the vessel into tissue of the patient.

In another aspect, a therapy delivery device includes: a catheter having a distal end and a proximal end; and an infusion device coupled to a proximal end of the catheter, the infusion device configured to inject an agent into or across a wall of a vessel.

In another aspect, a method for applying electrical stimulation using an endovascular transvascular technique includes: inserting a therapy delivery device comprising a stimulation device into a vessel of a patient; positioning the therapy delivery device in the vessel near nervous system tissue; and applying, from the stimulation device, stimulation therapy into or across a wall of the vessel to the nervous system tissue.

In another aspect, a method for aspirating bodily fluid using an endovascular transvascular technique includes: inserting a therapy delivery device comprising an aspiration device into a vessel of a patient; positioning the aspiration device in the vessel near a target area to be aspirated; puncturing, using the aspiration device, a wall of the vessel; and aspirating, using the aspiration device, a bodily fluid across a wall of the vessel to drain the bodily fluid.

In another aspect, a method for treating cancer using an endovascular transvascular technique includes: inserting a therapy delivery device comprising an infusion device into a vessel of a patient; positioning the therapy delivery device in the vessel near nervous system tissue; and applying, from the infusion device, a therapeutic into a wall of the vessel to treat the nervous system tissue and a neurologic condition.

In another aspect, a method for aspirating fluid using an endovascular transvascular technique includes: puncturing, by an aspiration device while in a vessel of a patient, a therapeutic, a wall of the vessel; and receiving, by the aspiration device, a bodily fluid across the wall of the vessel.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The terms “a” and “an” are defined as one or more unless this disclosure explicitly requires otherwise.

The term “about” as used herein can allow for a degree of variability in a value or range, for example, within 10%, within 5%, or within 1% of a stated value or of a stated limit of a range, and includes the exact stated value or range. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed implementation, the term “substantially” may be substituted with “within [a percentage] of” what is specified, where the percentage includes 0.1, 1, or 5 percent; and the term “approximately” may be substituted with “within 10 percent of” what is specified. The statement “substantially X to Y” has the same meaning as “substantially X to substantially Y,” unless indicated otherwise. Likewise, the statement “substantially X, Y, or substantially Z” has the same meaning as “substantially X, substantially Y, or substantially Z,” unless indicated otherwise. Unless stated otherwise, the word or as used herein is an inclusive or and is interchangeable with “and/or.” To illustrate, A, B, or C includes: A alone, B alone, C alone, a combination of A and B, a combination of A and C, a combination of B and C, or a combination of A, B, and C. Similarly, the phrase “A, B, C, or a combination thereof” or “A, B, C, or any combination thereof” includes: A alone, B alone, C alone, a combination of A and B, a combination of A and C, a combination of B and C, or a combination of A, B, and C.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any implementation of any of the systems, methods, and article of manufacture can consist of or consist essentially of—rather than comprise/have/include—any of the described steps, elements, or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, the term “wherein” may be used interchangeably with “where”.

Further, a device or system that is configured in a certain way is configured in at least that way, but it can also be configured in other ways than those specifically described. The feature or features of one implementation may be applied to other implementations, even though not described or illustrated, unless expressly prohibited by this disclosure or the nature of the implementations.

Some details associated with the implementations are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the implementation in the view.

FIG. 1 illustrates a block diagram of a system for applying transvascular therapy, such as for use in treating cerebral vasospasms.

FIG. 2 is a flowchart illustrating an example of a method of treating cerebral vasospasms.

FIG. 3 is a flowchart illustrating another example of a method of treating cerebral vasospasms.

FIG. 4 is a flowchart illustrating an example of a method of transvascular aspiration.

FIG. 5 is a flowchart illustrating an example of a method of transvascular cancer treatment.

FIG. 6 is a flowchart illustrating yet another example of a method of treating cerebral vasospasms and cancer.

FIG. 7 is a flowchart illustrating yet another example of a method of transvascular aspiration.

FIG. 8 illustrates examples of inflatable devices of the system for applying transvascular therapy.

FIG. 9 illustrates examples of expandable devices of the system for applying transvascular therapy.

FIG. 10 illustrates examples of injection or puncture devices of the system for applying transvascular therapy.

FIG. 11 illustrates examples of inflatable and expendable devices of the system for applying transvascular therapy.

FIG. 12 is illustrates example configurations for inflatable and expandable devices of the system for applying transvascular therapy.

FIG. 13 illustrates images of normal and abnormal vessels.

FIG. 14 is a diagram of an example of nervous system tissue.

FIG. 15 is a diagram of an example of a nervous system.

FIG. 16 is a diagram of an example of a nervous system tissue of a neck of a human.

FIG. 17 is images illustrating localization of human SCG for endovascular access.

FIG. 18 illustrates images of endovascular access to the human SCG.

FIG. 19 is an image of catheter in a vessel applying therapy to a ganglion.

FIG. 20 is an image of expanded view of the catheter of FIG. 19.

FIG. 21 is a photograph illustrating a Swine left neck dissection for the first the clinical trial.

FIG. 22 is a photograph illustrating Representative Regions of Interest for the first the clinical trial.

FIG. 23 illustrates images of Computed Tomography Perfusion Scans for the first the clinical trial.

FIG. 24 is a graph illustrating a Box and Whisker plot of CTp parameters in intracranial areas for the first the clinical trial.

FIG. 25 is a graph illustrating a Box and Whisker plot of CTp parameters with SCG blockade in intracranial areas for the first the clinical trial.

FIG. 26 is an image illustrating a Representative Anterior-Posterior (AP) view of swine cerebral angiogram from the left ascending pharyngeal artery for the second the clinical trial.

FIG. 27 is an image illustrating a Representative Contrast Flow Measurements on Syngo iFlow of AP view of Swine Cerebral Angiogram for the second the clinical trial.

FIG. 28 illustrates representative cerebral DSA images (AP View) with left ascending pharyngeal artery contrast injection for the second the clinical trial.

FIGS. 29 and 30 are graphs illustrating Box and Whisker plots of Vessel Diameter and Contrast Flow through Intracranial Arteries for the second the clinical trial.

FIG. 31 illustrates multiple photographs taken during the study of repeated puncturing of the carotid artery.

### DETAILED DESCRIPTION

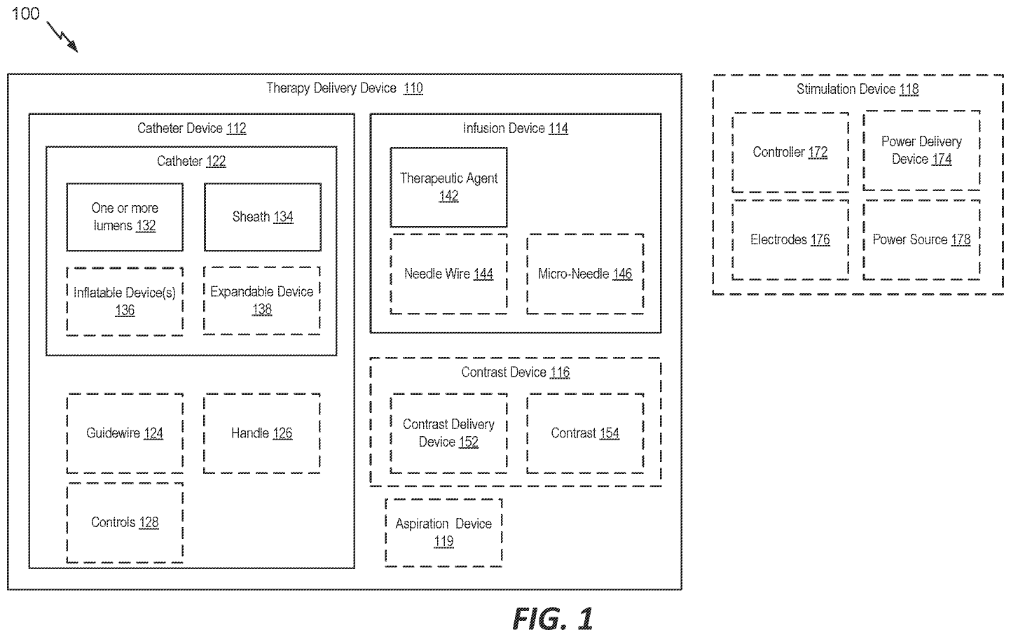

FIG. 1 is a block diagram of an example of a system 100 for use in providing transvascular therapies. These transvascular therapies may be used to treat cerebral vasospasms, hemorrhages (e.g., subarachnoid hemorrhage), cancer, and other conditions or diseases. System 100 may be used to apply therapies from within a patient's blood vessel to a wall of the vessel or across the wall of the vessel into tissue or cavities surrounding the vessel. For example, medications and/or stimulation therapy (activating or inhibiting) can be delivered from within a cerebral or cardiac artery to nearby nervous system tissue to inhibit a sympathetic nervous system response. In a particular implementation, the therapy may be applied to a cervical ganglion or to a pocket of space or fluid, such as cerebral spinal fluid. As another example, system 100 may be used to aspirate or drain fluid from a sensitive area, such as the brain or a subdural space. In a particular illustration, cerebral spinal fluid may be drained. As described herein, system 100 may be utilized for transvascular treatments and application of medications for reducing vasoconstriction and increasing blood flow. Such treatments and applications enable the successful localized treatment of sensitive areas, such as the neck and brain, including the treatment for cerebral vasospasms.

System 100, such as a transvascular therapy delivery system, includes a therapy delivery device 110 including a catheter device 112 and an infusion device 114. The catheter device 112 may include or correspond to a transvascular catheter or catheter system, such as an endovascular catheter, a peripheral vessel catheter, a cerebral vessel catheter, a carotid vessel catheter, an artery catheter, a vein catheter, a vessel catheter, as illustrative, non-limiting examples. The catheter device 112 is configured to be inserted into a body of a patient. The catheter device 112 may be or have an over-the-wire configuration or rapid exchange configuration, as illustrative, non-limiting examples.

The catheter device 112 includes a catheter 122. The catheter 122 includes one or more lumens 132 and a sheath 134. The one or more lumens 132 are defined by and/or included within the sheath 134. The catheter 122 may optionally include one or more inflatable devices 136, such as a first inflatable device and a second inflatable device, as illustrated in the example of FIG. 1, coupled to or positioned at the distal end. In other examples, the catheter 122 includes a single inflatable device (e.g., inflatable device 136) or includes no inflatable devices. The inflatable device(s) 136 may include or correspond to an inflatable balloon. The inflatable device(s) 136 may be inflated in vivo. The inflatable device(s) 136 may be configured to position and restrain the catheter device 112, apply a therapy, isolate a segment of a vessel and block blood flow, or a combination thereof. For example, the inflatable device(s) 136 may position and/or actuate a therapy device (e.g., infusion device 114 or stimulation device 118) as further described herein.

The catheter 122 may optionally include one or more expandable devices 138, such as a stent or a balloon, coupled to or positioned at the distal end. The catheter 122 may include the expandable device 138 in addition to or in the alternative of the inflatable device or devices 136. The expandable device 138 may be expanded in vivo. The expandable device 138 may be configured to position and restrain the catheter device 112, apply a therapy, isolate a segment of a vessel and block blood flow, or a combination thereof. For example, the expandable device 138 may actuate a therapy device (e.g., infusion device 114 or stimulation device 118) as further described herein.

In some implementations, catheter device 112 may, but need not, include one or more of a guidewire 124, a handle 126, controls 128, or a combination thereof. For example, the catheter 122 may be advanced along a guidewire 124 and be controlled (e.g., guided or steered) by the handle 126 and/or controls 128 at the proximal end in some implementations. The guidewire 124 may be inserted into a blood vessel of the patient directly or into an access or opening in the blood vessel.

In some implementations, such as implementations which involve accessing vessels of the brain and/or treating brain tissue transvascularly, a catheter of therapy delivery device may include one or more additional features. For example, the catheter may have a variable stiffness along a length of the catheter. To illustrate, the catheter may include one or more reinforcement sections. In some implementations, the catheter includes a coiled section, such as section of coiled wire (metal wire) or a polymer with a coil reinforced shaft to reinforce the catheter and help guide the catheter. Additionally or alternatively, the catheter may use different materials, different thicknesses, and/or different diameters along a length of the catheter shaft to achieve a desired stiffness or change in stiffness.

As another example, the catheter may have an external coating. To illustrate, an exterior of the catheter may have a hydrophilic coating to reduce surface friction and enhance trackability of the catheter. In a particular implementation, a polymer catheter has a surface coating of a hydrophilic material. As another illustration, a portion of the catheter or therapy delivery device may be coated in a drug, such as a drug eluting stent. Such features may enable or provide additional benefits for treating areas of the brain from cerebral vessels or the venous sinus.

The infusion device 114 may be coupled to a distal end of the therapy delivery device 110. For example, the infusion device 114 may be coupled to the distal end of the catheter device 112. To illustrate, in some implementations, the infusion device 114 is coupled to the distal end of the catheter 122, such as to the inflatable device(s) 136. In some implementations, the infusion device 114 is coupled to multiple inflatable devices. For example, the infusion device 114 may include multiple needles or inject instruments, and one or more such needles or inject instruments may be coupled to each inflatable device. Needles may protrude perpendicular or at other angles from a longitudinal or axial axis of the device.

Alternatively, in other implementations, the infusion device 114 is coupled to the catheter device 112. For example, the infusion device 114 may be coupled to the sheath 134 and extended or advanced away from the sheath 134 and into the wall of the vessel, such as by a transverse orientation with respect to a longitudinal direction of the catheter device 112 and vessel. As another example, the infusion device 114 may be coupled to the catheter device 112 and advanced through a corresponding lumen of the one or more lumens 132. The corresponding lumen (e.g., an injection or infusion lumen) may include an aperture in the sheath 134 near the distal end of the catheter device 112 to enable a portion (e.g., a delivery mechanism or needle) of the infusion device 114 to extend outside of the catheter 122 and into (or through) the wall of the vessel.

The infusion device 114 may include or correspond to needle wire 144 or micro-needle 146 based device, as illustrative, non-limiting examples. The infusion device 114 may include a delivery structure (e.g., needle, microneedle, etc.) and a therapeutic agent 142. The therapeutic agent 142 (also referred to herein as a therapeutic) may be loaded into the infusion device 114 and/or therapy delivery device 110 prior to insertion in the body. Alternatively, the infusion device 114 may receive the therapeutic agent 142 after insertion in the body (in vivo). The infusion device 114 is configured to apply, such as inject or release the therapeutic agent 142 via the delivery structure. The therapeutic agent 142 may include a medication, a gel, a slow release delivery or long lasting medication (e.g., a slow release drug polymer), a sclerosis agent, a biologic, DNA, RNA, a biosimilar, a cancer drug, etc. As an illustrative, non-limiting example, the therapeutic agent 142 is an anesthetic (e.g., lidocaine). In some implementations, the therapeutic agent 142 may include multiple types of therapeutics, such as medication and a biologic.

Additionally, or alternatively, system 100 may include an aspiration device 119 similar to infusion device 114, such as having one or more similar components. For example, the infusion device 114 may also be configured to puncture a wall of a vessel to remove fluid outside of or around the vessel prior to injecting or infusing a therapeutic into or across the wall of the vessel. As another example, the system 100, such as the therapy delivery device 110 thereof of another device, may include an aspiration device 119 as an alternative to or separate from the infusion device 114. In some such implementations, the therapy delivery device 110 may be an aspiration therapy device and the aspiration device 119 thereof may drain fluid to provide the therapy, and optionally without providing a therapeutic, such as when the therapy delivery device 110 does not include the infusion device 114.

In some implementations, system 100 may, but need not, include a contrast device 116, such as contrast delivery mechanism. In some implementations, the contrast device 116 is fluoroscopy or CT based contrast delivery device. The contrast device 116 115 may include a contrast delivery device 152, contrast 154, or a combination thereof. The contrast may be injected or released from the contrast delivery device 152 to enable position of one or more components of the therapy delivery device 110. For example, the contrast may be used with radiographic imaging to determine a position of a particular component (e.g., balloon or needle) of the therapy delivery device 110. In some implementations, contrast can also be mixed with the therapeutic to improve visibility of the injection of the therapeutic. Additionally, or alternatively, contrast can also be injected across the vessel wall to confirm position of the device prior injection and/or aspiration as a therapeutic intervention. The therapy delivery device 110 may include a marker (e.g., a radiographic marker) in addition to or in the alternatively of the contrast device 116 or using contrast 154.

In some implementations, system 100 may, but need not, include a stimulation device 118, such as an electrical therapy stimulation device. In some implementations, the stimulation device 118 is an electrode based device and configured to provide electrical signals to modulate the surrounding tissue, such as by activation and/or inhibition of the target tissue, surrounding tissue, or both. In other implementations, the stimulation device 118 is configured to generate or transfer heat to provide heat based therapy and/or ablate tissue.

The stimulation device 118 may include a controller 172, a power delivery device 174, one or more electrodes 176, a power source 178, or a combination thereof. The stimulation device 118 is configured to transvascularly provide an electrical signal via the one or more electrodes 176 to an area near the nervous system tissue or to the tissue itself. The electrical stimulation can be activating or inhibiting of the target tissue depending on the electric stimulation protocol. Alternatively, the stimulation device 118 may generate or transfer heat to provide heat therapy or ablate tissue to provide neuromodulation therapy.

The controller 172 may include a processor, a memory, a network interface, or a combination thereof. In some implementations, the system 100 may include one or more other components such as a display, one or more input/output (I/O) devices, or the like. The processor may be coupled to the memory, the network interface, the display, or the one or more I/O devices. The processor may be a general purpose computer system (e.g., a personal computer (PC), a server, or a tablet device), a central processing unit (CPU), a special purpose processor platform (e.g., application specific integrated circuit (ASIC) or system on a chip (SoC)), or other computing circuitry. The processor may include one or more processors, such as a baseband processor, an application processor, a graphics processor, or a combination thereof, as illustrative, non-limiting examples.

The processor may be configured to provide therapy. In other implementations, processor may be configured to determine or adjust the therapy, as described herein. For example, the processor may be configured to control the stimulation device 118. In other examples, the processor may be configured to control the infusion device 114.

The memory includes instructions and optionally one or more data sets. As an illustrative, non-limiting example, at least one of the data sets includes or corresponds to a patient profile that is associated with a patient. The patient profile can include one or more items or variables, such as, for example, past or current measurements, thresholds, ranges, or calculated values, as illustrative, non-limiting examples. To further illustrate, the patient profile may include various medical information of the patient, such as a therapy history, a nervous system level of activity history, a target therapy range, a combination thereof, or the like, as illustrative, non-limiting examples.

The data stored in the patient profile may be accessible to or accessed by the processor. For example, the processor may obtain, update, or store data. Additionally, or alternatively, the processor may select data from the patient profile to use in calculations or other operations performed by the processor or another device.

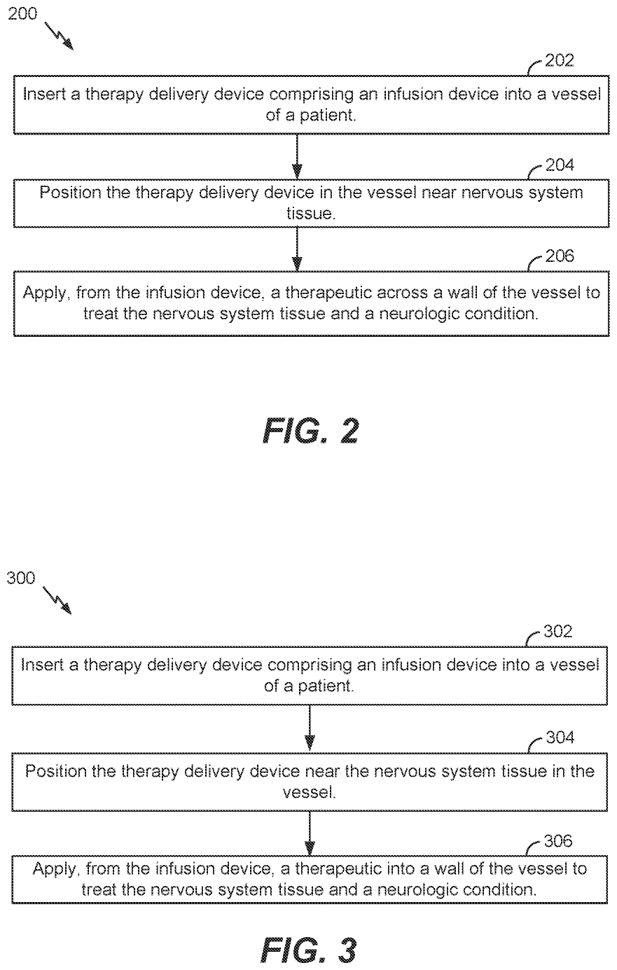

During operation, system 100 may apply one or more therapies to nervous system tissue to perform neuromodulation. The neuromodulation enables treatment of neurological disorders including cerebral vasospasms. Operation of system 100 is described with reference to FIGS. 2-7.

Accordingly, system 100 advantageously provides a new targeted approach and transvascular therapies for neurological issues, including cerebral vasospasms and a corresponding device for providing the therapy. The system 100 can use an endovascular approach and apply transvascular therapy directly to nervous system tissue or a region associated with the nervous system tissue such that the therapy can affect (e.g., inhibit) certain nervous system responses and/or cause certain benefits. Specifically, the therapy can inhibit or modulate sympathetic response and increase blood flow and/or reduce vasoconstriction. Thus, a patient care provider may be provided with additional treatment options which are more targeted and less invasive as compared to conventional nervous system treatments.

Referring to FIGS. 2 and 3, methods of treating cerebral vasospasms are illustrated. For example, each of the methods of FIGS. 2 and 3 may be performed by system 100, such as by therapy delivery device 110, a patient care provider, or both.

Referring to FIG. 2, a method 200 of treating cerebral vasospasms includes inserting a therapy delivery device comprising an infusion device into a vessel of a patient, at 202. For example, the therapy delivery device 110 of FIG. 1 may be inserted into a vessel of a patient. In some implementations, the catheter device 112 and infusion device 114 are coupled together and advanced together into a cerebral or carotid artery of the patient.

The method 200 also includes positioning the therapy delivery device in the vessel near the nervous system tissue, at 204. For example, a distal end of the catheter 122 is advanced through the vessel until the distal end is proximate or adjacent to tissue that is targeted for therapy, such as a SC ganglion and/or surrounding nervous system tissue. The nervous system tissue may include sympathetic and/or para-sympathetic nervous system tissue and/or nearby receptors of other tissue which can control or active nervous system tissue.

Positioning the therapy delivery device near the tissue to be treated may include one or more additional steps as described herein. For example, positioning the therapy delivery device may include using image assisting techniques, such as radiography, ultrasound, fluoroscopy, angioscopy, etc. As another example, positioning the therapy delivery device may include activating the therapy delivery device. To illustrate, a balloon or stent may be expanded, a needle may be moved into a delivery position, etc. In some implementations, positioning or anchor balloons may be utilized to set the therapy delivery device in place. As yet another example, an area may be drained of fluids, aspirated, prior to injection, such as described with reference to FIG. 5. Alternatively, the drainage or aspiration of fluid may be done as the therapy alone (for example drainage of subdural hematoma), and independent of providing other therapy (e.g., electrical stimulation or infusion of a therapeutic).

The method 200 also includes applying, from the infusion device, a therapeutic across a wall of the vessel into tissue of the patient to treat the nervous system tissue and a neurologic condition, at 206. For example, a micro-needle 146 of the infusion device 114 injects the therapeutic agent 142 across the wall into tissue or voids near a ganglion. In some implementations, the therapeutic agent 142 includes a local anesthetic, such as an amide or ester group local anesthetic. As an illustrative example, lidocaine may be injected. The injection of the therapeutic agent applies therapy to the nervous system. This therapy may cause a reduction is vasoconstriction, an increase in blood flow, or both, and may thus treat cerebral vasospasms.

Referring to FIG. 3, a method 300 of treating cerebral vasospasms includes inserting a therapy delivery device comprising an infusion device into a vessel of a patient, at 302. For example, the therapy delivery device 110 of FIG. 1 may be inserted into a vessel of a patient. In some implementations, the catheter device 112 and infusion device 114 are coupled together and advanced together into a cerebral or carotid artery of the patient.

The method 300 also includes positioning the therapy delivery device in the vessel near the nervous system tissue, at 304. For example, a distal end of the catheter 122 is advanced through the vessel until the distal end is proximate or adjacent to tissue that is targeted for therapy, such as a SC ganglion and/or surrounding nervous system tissue. Positioning the therapy delivery device near the tissue to be treated may include one or more additional steps as described herein and with reference to FIG. 2.

In some implementations, the method 300 also includes applying, from the infusion device, a therapeutic into a wall of the vessel to treat the nervous system tissue and a neurologic condition, at 306. For example, a micro-needle 146 of the infusion device 114 injects the therapeutic agent 142 into the wall of the vessel itself, at a point near a ganglion. In some implementations, the therapeutic agent 142 includes a local anesthetic, such as an amide or ester group local anesthetic. As an illustrative example, lidocaine may be injected. The injection of the therapeutic agent applies therapy to the nervous system. This therapy may cause a reduction is vasoconstriction, an increase in blood flow, or both, and may thus treat cerebral vasospasms.

As compared to FIG. 2, FIG. 3 illustrates a method of treatment where the therapy is applied to the wall of vessel. In other implementations, the treatment may be applied to the wall only, across the wall only, or both to and across the wall.

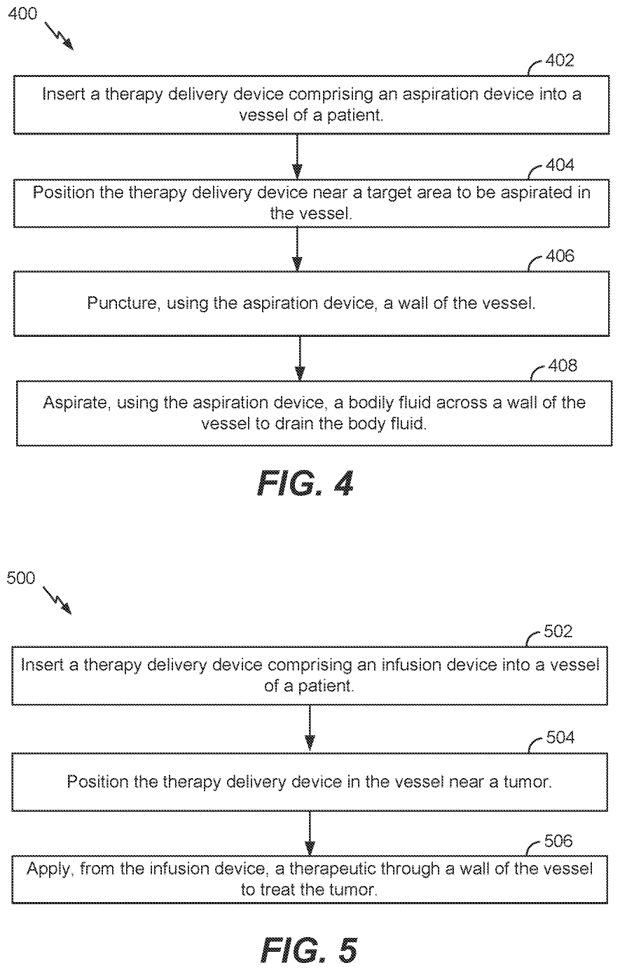

Referring to FIGS. 4 and 5, methods of aspirating and treating cancer transvascularly are illustrated. For example, each of the methods of FIGS. 4 and 5 may be performed by system 100, such as by therapy delivery device 110, a patient care provider, or both.

Referring to FIG. 4, a method 400 of transvascularly aspirating bodily fluid includes inserting a therapy delivery device comprising an aspiration device into a vessel of a patient, at 402. For example, the therapy delivery device 110 of FIG. 1 may be inserted into a vessel of a patient. In some implementations, the catheter device 112 and aspiration device 119 and infusion device 114 are coupled together and advanced together into a cerebral or carotid artery of the patient.

The method 400 also includes positioning the therapy delivery device near a target area to be aspirated in the vessel, at 404. For example, a distal end of the catheter 122 is advanced through the vessel until the distal end is proximate or adjacent to a bodily fluid that is to be drained or aspirated.

The method 400 also includes puncturing, using the aspiration device, a wall of the vessel of the patient, at 406. For example, the aspiration device 119 or the infusion device 114 punctures the wall of the vessel with a needle. To illustrate, a balloon or stent is expanded to release a needle from a retracted state and drive the needle into and through the wall near the target area. In some implementations, the balloon can be used to obstruct blood flow during the therapy. Alternatively, a tubular balloon can be used to allow blood to pass through the tube while still driving a needle into and through the vessel wall near the target area.

The method 400 also includes aspirating, by the aspiration device, a bodily fluid across the wall of the vessel to drain the bodily fluid, at 408. For example, an aspiration device or the infusion device 114 receives bodily fluid (e.g., blood, cerebrospinal fluid, chronic hematoma, interstitial fluid, lymphatic fluid, etc.) across the wall of the vessel itself. In some implementations, the aspiration device 119 is used to remove excess fluid in the brain, such as to remove a SAH or relieve pressure from a SAH or subdural hematoma, from a cerebral blood vessel. Additionally, or alternatively, the aspiration device 119 may be used prior to injection of a therapeutic by an infusion device. In some implementations, the aspiration device 119 is separate from the infusion device and/or the therapy delivery device. In other implementations, the aspiration device 119 may include or be part of the infusion device 114. In a particular implementation, therapy may be provided to the aspirated area by a therapy delivery device, similar the method of FIGS. 2 and 3.

Referring to FIG. 5, a method 500 of treating cancer includes inserting a therapy delivery device comprising an infusion device into a vessel of a patient, at 502. For example, the therapy delivery device 110 of FIG. 1 may be inserted into a vessel of a patient. In some implementations, the catheter device 112 and infusion device 114 are coupled together and advanced together into a cerebral or carotid artery of the patient.

The method 500 also includes positioning the therapy delivery device near a tumor in the vessel, at 504. For example, a distal end of the catheter 122 is advanced through the vessel until the distal end is proximate or adjacent to cancerous tissue that is targeted for therapy, such as a tumor.

In some implementations, the method 500 also includes applying, from the infusion device, a therapeutic through a wall of the vessel to treat the tumor, at 506. For example, a micro-needle 146 of the infusion device 114 injects the therapeutic agent 142 across the wall of the vessel itself, at a point near the tumor or into the tumor directly to treat the tumor. The tumor may reside in a sensitive area that is not otherwise suitable for targeted therapies, such as radiation or surgery. As illustrative examples, cancer in the brain, face, and neck areas may be treated. Additionally, localized treatment of the cancer may have less systematic complication than other non-or less-targeted therapies, such as chemotherapy. The therapeutic cancer agent may include or correspond to a sclerosing or embolic material in some implementations.

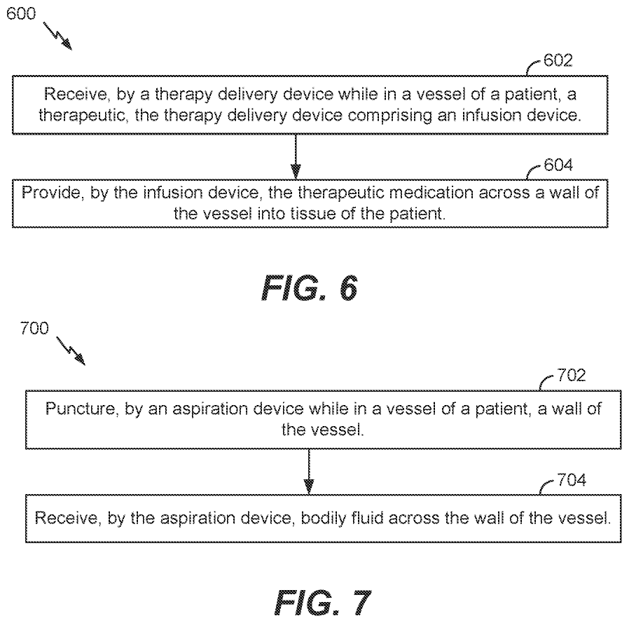

FIG. 6 illustrates a method of treating cerebral vasospasms or tumors transvascularly. For example, the methods of FIG. 6 may be performed by system 100, such as by therapy delivery device 110.

Referring to FIG. 6, a method 600 of treating cerebral vasospasms or tumors transvascularly includes receiving, by a therapy delivery device while in a vessel of a patient, a therapeutic, the therapy delivery device comprising an infusion device, at 602. For example, a reservoir in or associated with the infusion device 114 receives the therapeutic agent 142. To illustrate, the reservoir may receive the therapeutic agent 142 from a syringe or other device prior to insertion in the body. As another illustration, the reservoir or the needle itself may receive the therapeutic agent 142 after insertion into the body and positioning near a ganglion or tumor.

The method 600 also includes providing, by the infusion device, the therapeutic medication across a wall of the vessel into tissue of the patient, at 604. For example, a micro-needle 146 of the infusion device 114 injects the therapeutic agent 142 into the wall of the vessel itself, at a point near the ganglion, or through the wall into the area outside of the vessel or into nearby nervous system tissue or the ganglion itself, or into or nearby a tumor. In some implementations, the therapeutic agent 142 includes a local anesthetic, such as an amide or ester group local anesthetic. As an illustrative example, lidocaine may be injected. The injection of the therapeutic agent applies therapy to the nervous system. This therapy may cause a reduction is vasoconstriction, an increase in blood flow, or both, and may thus treat cerebral vasospasms. In some other implementations, the therapeutic agent 142 includes a cancer drug. The injection of the cancer drug applies therapy to the tumor itself. This therapy may cause a reduction in tumor size, elimination of the tumor, reduced growth rate of the tumor, etc. The therapeutic cancer agent may include or correspond to a sclerosing or embolic material in some implementations.

FIG. 7 illustrates a method of aspirating boldly fluid transvascularly. For example, the methods of FIG. 7 may be performed by system 100, such as by therapy delivery device 110. Referring to FIG. 7, a method 700 of aspirating boldly fluids includes puncturing, by an aspiration device while in a vessel of a patient, a wall of the vessel, at 702. For example, the aspiration device 119 or the infusion device 114 punctures the wall of the vessel with a needle. To illustrate, a balloon or stent is expanded to release a needle from a retracted state and drive the needle into and through the wall.

The method 700 also includes receiving, by the aspiration device, the bodily fluid across the wall of the vessel, at 704. For example, an aspiration device or the infusion device 114 receives bodily fluid (e.g., blood, interstitial fluid, liquid hematoma, cerebrospinal fluid, lymphatic fluid, etc.) across the wall of the vessel itself. In some implementations, the aspiration device 119 is used to remove excess fluid in the brain, such as to remove a SAH or relieve pressure from a SAH. Additionally, or alternatively, the aspiration device 119 may be used prior to injection of a therapeutic by an infusion device. In some implementations, the aspiration device 119 is separate from the infusion device and/or the therapy delivery device. In other implementations, the aspiration device 119 may include or be part of the infusion device 114.

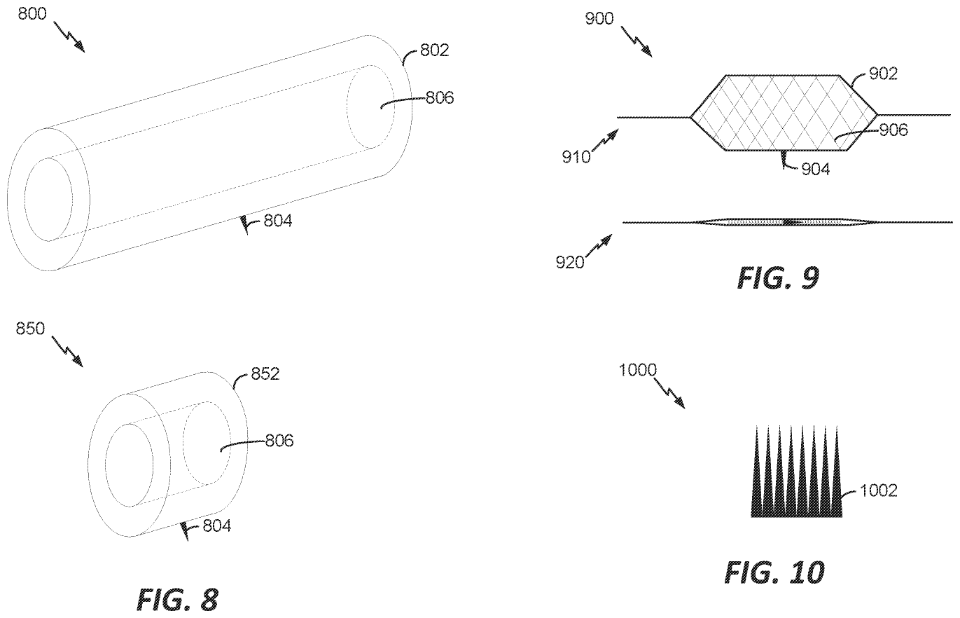

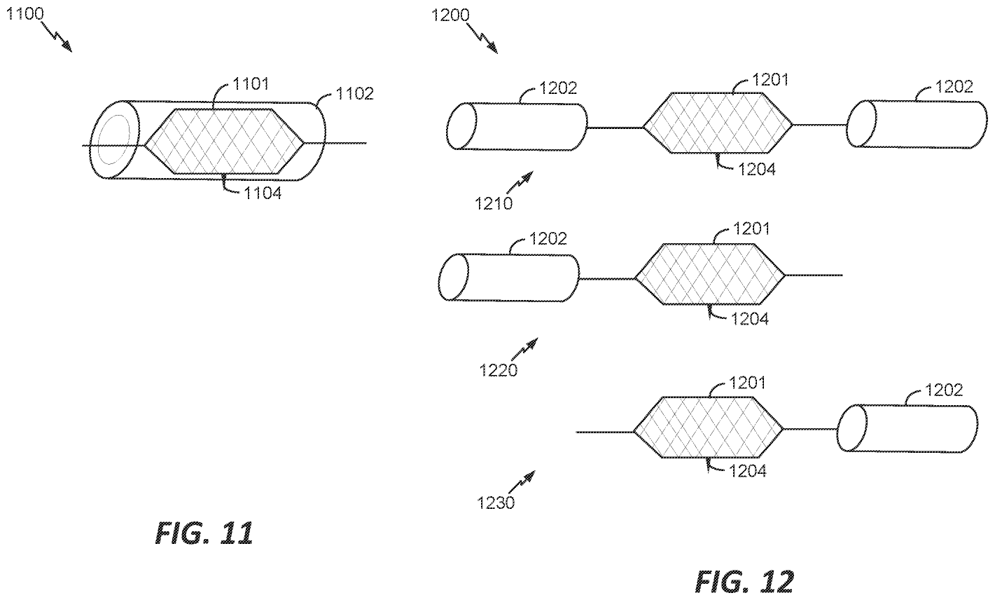

FIGS. 8-12 illustrates example configurations of a therapy delivery device, and components thereof. In FIGS. 8 and 9, examples of inflatable devices and expandable devices are illustrated. FIG. 10 illustrates examples of puncturing elements of an injection or aspiration device. FIGS. 11 and 12 illustrate example configurations of inflatable deices and expandable devices for a therapy delivery device.

Referring to FIGS. 8 and 9, different inflatable and/or expandable elements are illustrated for the therapy delivery devices described herein. The different inflatable and/or expandable elements may include an injection or infusion element, such as a needle, a needle wire, a microneedle, etc. The inflatable and/or expandable elements may be used as an inflatable device of FIG. 1 and/or as an infusion device of FIG. 1.

As illustrated in FIG. 8, in a first implementation 800, a hollow balloon 802 includes an retractable needle 804. The retractable needle 804 is configured to transition from a first position to a second position upon inflation of the hollow balloon. The hollow balloon includes a central aperture 806 for blood to flow while the balloon 802 is inflated. This enables the balloon 802 to occlude an injection area/delivery area, while not occluding blood flow. Such a hollow balloon 802 may provide advantages over conventional spherical balloons which occlude blood flow in the vessel.

As illustrated in FIG. 8, in a second implementation 850, a shorter hollow balloon 852 is also illustrated. As compared to the first implementation, the second implementation is shorter in length (a longitudinal direction of in vessel) and has more of a torus (doughnut) shape.

As illustrated in FIG. 9, in a third implementation 900, an expandable stent 902 includes a retractable needle 904. Similar to the first implementation, the retractable needle 904 is configured to transition from a first position to a second position upon expansion of the expandable stent, as shown in a compressed shape 910 and an expanded shape 920. The expandable stent may include a series of struts 906 (e.g., metal supports) which enable the stent to be transitioned between the first and second position. The expandable stent 902 may be transitioned between the first and second position based on applying force to a portion of the stent, such as a base thereof, extension from a sheath, or a combination thereof. To illustrate, the expandable stent 902 may be biased towards an expanded state or my transition to an expanded state based on temperature (e.g., by using a shape memory alloy). The expandable stent 902 may be restrained in a closed or unexpanded state by a sheath or cover, and the expandable stent 902 may transition to the expanded shape 920 after removal of the restraining force, such as by removal of the sheath or cover.

Additionally, or alternatively, the expandable stent 902 may include a wire mesh. The wire mesh may be transitioned between the first and second position based on moving the stent outside of a sheath or an end or opening of the catheter. Similar to the hollow annular portion of the hollow balloon, the expandable stent allows for vessel blow flow while in an expanded state and delivering a therapeutic.

As compared to the hollow balloons 802 and 852, the expandable stent 902 of FIG. 9 may offer greater blood vessel flow and may not even occlude the injection area. Alternatively, the expandable stent 902 may include a small solid or inflatable portion to occlude the injection area/delivery area, while not occluding blood flow. Such an expandable stent may provide advantages over conventional transvascular delivery devices.

In a fourth implementation 1100, an expandable stent 1101 includes a retractable needle or needles 1104 and an inflatable balloon 1102, as illustrated in FIG. 11. Similar to the third implementation, the fourth implementation includes the expandable stent 1101 and retractable needles 1104. However, in the fourth implementation 1100, the inflatable balloon 1102 is an annular hollow balloon positioned around the expandable stent 1101 for delivery area or transvascular wall occlusion. As shown in FIG. 11, the retractable needle or needles 1104 may be operable through the inflatable balloon 1102. Alternatively, the retractable needle or needles 1104 may be coupled to the inflatable balloon 1102 and operate based on inflation or expansion of inflatable balloon 1102, as shown in FIG. 8.

As shown in FIG. 12, a therapy device 1200 as described herein may include a stent 1201 (e.g., 902 or 1101) and one or more other inflatable devices 1202 (e.g., 802, 852, 1102), in addition to the devices and implementations shown in the previous FIGS. of 8, 9, and 11. For example, one or more inflatable devices 1202 (e.g., 802, 852, 1102) or expandable devices (e.g., 902 or 1101) may be used to secure, position, and/or occlude blood flow from portions of the vessel while operating the stent 1201. To illustrate, balloons and/or stents as described herein may be used upstream and/or downstream from an injection area to occlude blood flow upstream and/or downstream of the injection area, as shown in examples 1210, 1220, and 1230. In one particular implementation, balloons are used upstream and downstream from an expandable stent to prevent or limit blood flow and optionally limit blood near the injection area. In another particular implementation, blood flow may be occluded downstream (with reference to blood flow) to reduce blood pressure downstream or increase pressure upstream. Alternatively, blood flow may be occluded upstream (with reference to blood flow) to reduce blood pressure upstream or increase pressure downstream. In another implementation, a stent may be used downstream and opposite a balloon upstream to reduce blood pressure downstream while limiting blood near the injection area.

Although a single needle is illustrated in many of the implementations in FIGS. 8, 9, 11, and 12, in other implementations multiple needles 1002 may be used or other injection devices may be used as illustrated in FIG. 10. For example, retractable needles (e.g., 804 or 904) may be placed on multiple sides of a stent or balloon. As another example, a microneedle (or series of small needles) may be placed on one or more portions of an exterior of the balloon or stent. As yet another example, the balloon or stent may not have an injection device, but may be configured to receive one. To illustrate, a wire needle may be guided through the balloon or stent to deliver the therapeutic.

Although the needles are illustrated in a perpendicular orientation or 90 degree angle with respect to a base or surface of the stent or balloon in FIGS. 8, 9, 11, and 12, in other implementations the needle may be arranged at another angle (e.g., not perpendicular) or be adjustable. An adjustable needle may be activated or controlled separate from expansion or movement of the stent or balloon, and may include two or more positions, such as two more injection positions or non-retraced positions.

Although the needles are illustrated as a single size in FIGS. 8, 9, 11, and 12, in other implementations the needle may have different sizes, such as longer, shorter, wider, narrower, etc. Bigger needles may be more suitable for veins, lower pressure vessels, and/or larger vessels. Additionally, longer needles may be used to deliver therapy deeper into the body or further into tissue. Smaller needles may be more suitable for arteries, higher pressure vessels, smaller vessels, and/or providing therapy into or near the wall of the vessel.

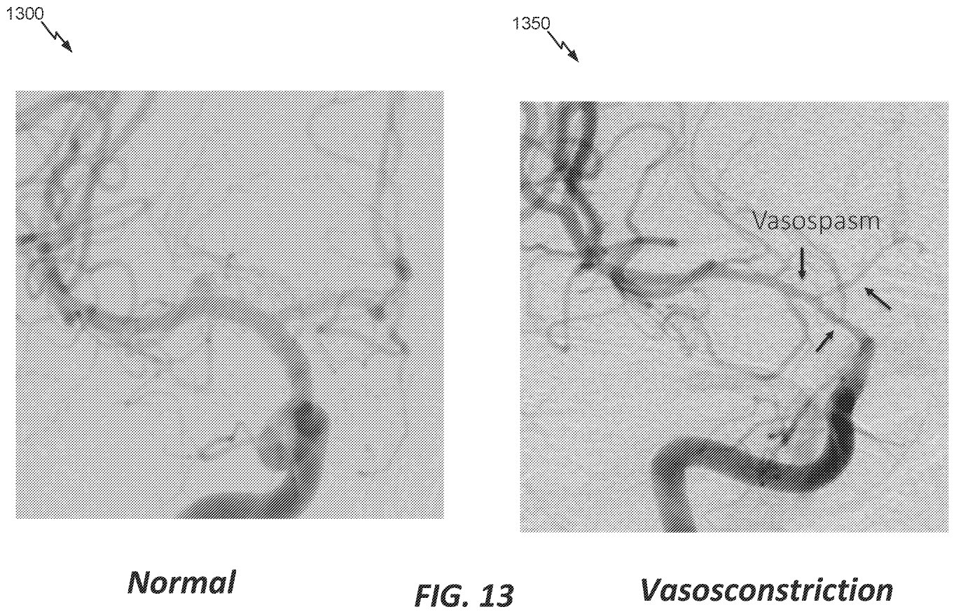

FIG. 13 is a diagram of examples of cerebral blood vessels. Referring to FIG. 13, an illustrative representation of normal cerebral blood vessels and of abnormal cerebral blood vessels are illustrated. The diagram 1300 illustrates normal cerebral blood vessels for reference. The diagram 1350 illustrates cerebral blood vessels which are constricted. As an illustrative example, the cerebral blood vessels may be experiencing vasospasm. Vasospasm is a condition in which an venous or arterial spasm leads to vasoconstriction. This can lead to tissue ischemia and tissue death. Cerebral vasospasm may arise in the context of subarachnoid hemorrhage. Symptomatic vasospasm or delayed cerebral ischemia is a major contributor to post-operative stroke and death especially after aneurysmal subarachnoid hemorrhage. Vasospasm may appear after a subarachnoid hemorrhage, such as multiple days after the subarachnoid hemorrhage. Symptoms of a vasospasm can vary depending on the area of the body affected. Alternatively, the blood vessels may be experience vasoconstriction from another cause or pathology.

Cerebral vasospasm after aneurysm rupture is one of the most preventable causes of mortality. Every year 30,000 patients develop an aneurysmal subarachnoid hemorrhage (aSAH) in the United States. Two-thirds of these patients develop vasospasm.

In addition, there are roughly 300,000 traumatic brain injury (TBI) related hospitalizations in the US every year. Of these hospitalized TBIs, up to 60 percent of these have SAH, and up to 60 percent have cerebral vasospasms, even in the absence of SAH. For the particular case of cerebral vasospasm that occurs after aSAH, a patient generally has 25 percent longer hospital stays and 25 percent higher total incremental costs. These leads to an additional 25,000 to 40,000 dollars per patient and an extra 4 to 6 days in the hospital.

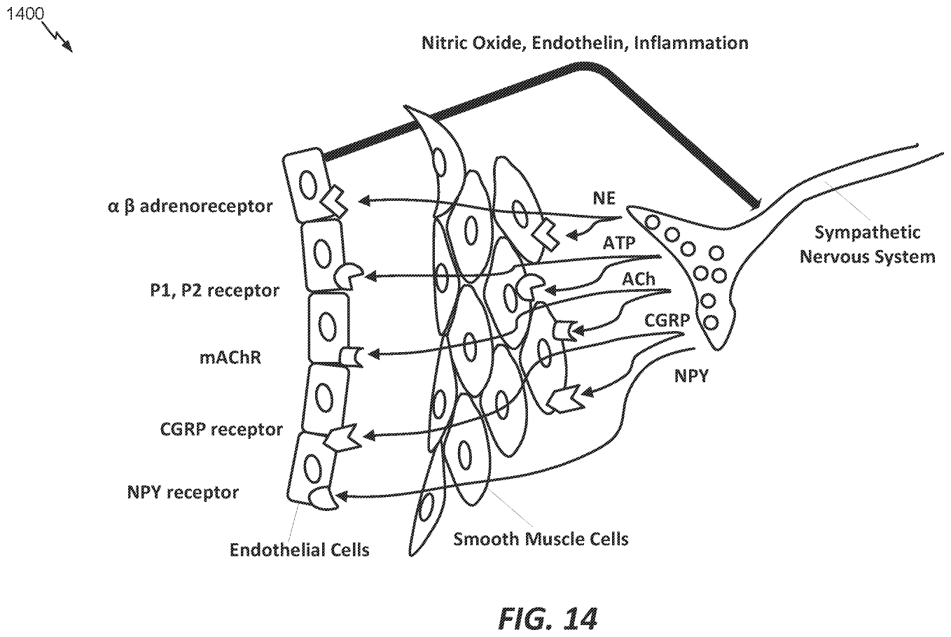

FIG. 14 is a diagram of an example of nervous system tissue. Referring to FIG. 14, an illustrative representation of nervous system tissue is illustrated. The diagram 1400 illustrates interactions between the sympathetic nervous system (e.g., sympathetic nervous system tissue) and surrounding tissues. As illustrated in FIG. 14, the sympathetic nervous system is proximate to and smooth muscle cells and endothelial cells. Receptors of such proximate tissue may activate the sympathetic nervous system and produce various responses.

The sympathetic nervous system is one of two divisions of the autonomic nervous system (involuntary nervous systems), along with the parasympathetic nervous system. The sympathetic nervous system is an extensive network of cells (e.g., neurons) that regulate the body's involuntary processes. The sympathetic nervous system, such as activation thereof, may control various responses of vasoconstriction, inflammation, leukocyte activation, oxidative stress, and increased levels of chemokines and cytokines as illustrative examples.

As described above, the therapy delivery devices described herein may deliver therapeutics to the sympathetic nervous system directly, such as by delivery into sympathetic nervous system tissue or in void which is in contact with sympathetic nervous system tissue. Additionally, or alternatively, the therapy delivery devices described herein may deliver therapeutics indirectly to the sympathetic nervous system, such as by delivery into nearby tissue which may influence or be “connected” to sympathetic nervous system. To illustrate, by injection of a therapeutic into or near endothelial or smooth muscle cells, the therapeutic may cause receptors of the endothelial or smooth muscle cells to active the sympathetic nervous system.

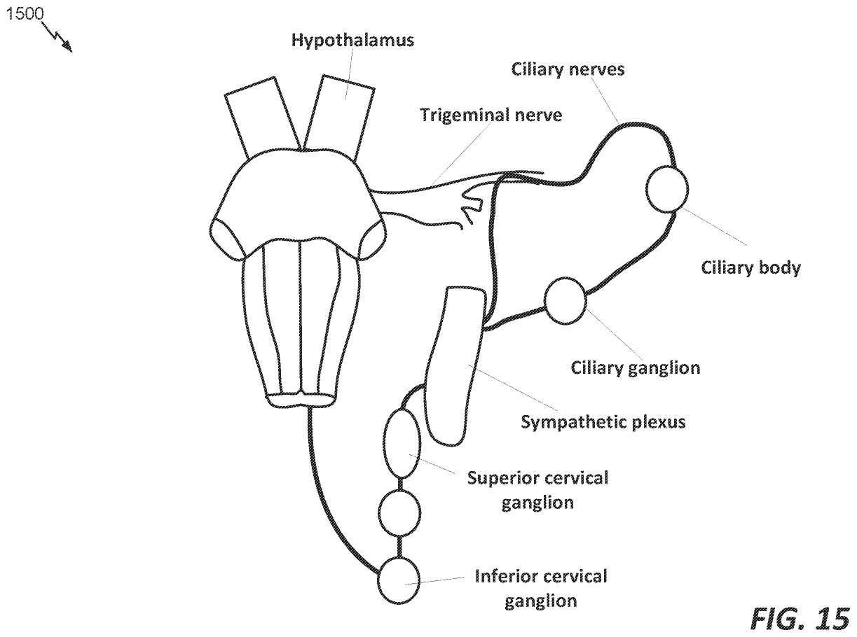

FIG. 15 is a diagram of an example of a nervous system. Referring to FIG. 15, an illustrative representation of a nervous system is illustrated. The diagram 1500 illustrates interactions couplings between elements (nerves, ganglions, glands, etc.) of a sympathetic nervous system. As illustrated in FIG. 15, the sympathetic nervous system includes multiple ganglions connected to each other and the hypothalamus via various nerves. Diagram 1500 illustrates inferior and superior cervical ganglions connected to the hypothalamus and a sympathetic plexus, such as a sympathetic plexus around the carotid artery. The sympathetic plexus is also connected to a trigeminal nerve which is coupled to the hypothalamus and other ganglions, such as the ciliary ganglion.

As described above, the therapy delivery devices described herein may deliver therapy and/or therapeutics to these elements of the sympathetic nervous system directly or indirectly, as described in reference to FIG. 14, to treat and influence one or more elements of the sympathetic nervous system. Although an example of the sympathetic nervous system is illustrated in FIG. 15, the therapy delivery devices described herein may deliver therapy and/or therapeutics to elements of the para-sympathetic nervous system in other implementations. Additionally or alternatively, the therapy delivery devices described herein may deliver therapy and/or therapeutics to other nervous system tissues and/or in other locations. As an example of another location, the therapy delivery devices described herein may deliver therapy and/or therapeutics to the subclavian artery and the therapy is configured to modulate the nerves of the brachial plexus. As an example of other tissue, the therapy delivery devices described herein may deliver therapy and/or therapeutics to nerves, axions, neurons, smooth muscle cells, endothelial cells, etc.

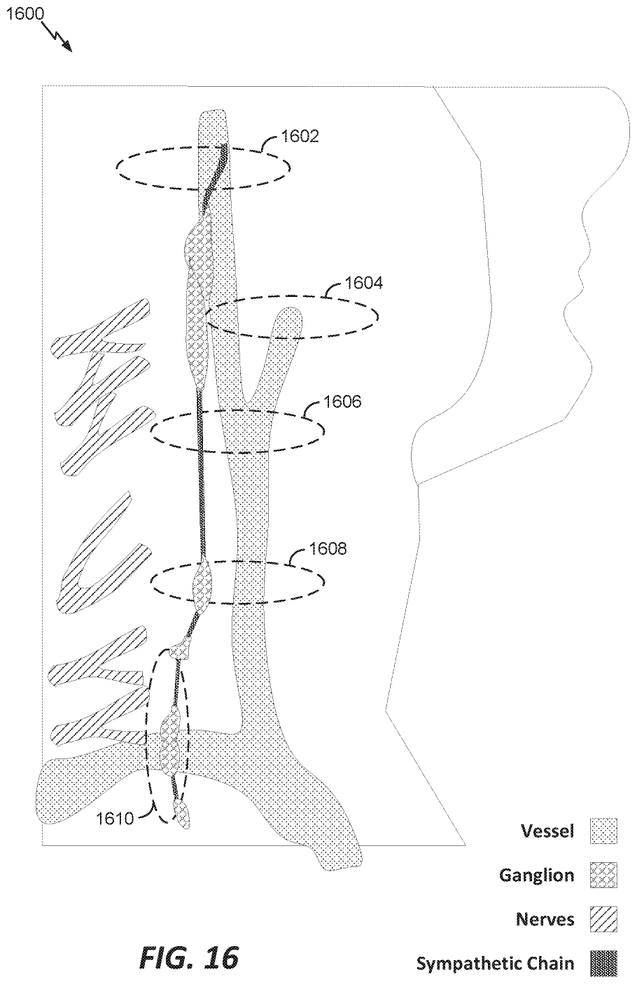

FIG. 16 is a diagram of an example of anatomy of a neck of a human. Referring to FIG. 16, an illustrative representation of tissue, such as nervous system tissue, and vessels (veins and arteries) of the neck of a human is illustrated. The diagram 1600 illustrates example injection areas or sites for the therapy delivery devices described herein to treat (e.g., activate, stimulate, inhibit, modulate, etc.) the nervous system tissue. The example injection areas or sites are shown with reference to vessels, cervical spinal nerves, and the sympathetic chain (including ganglions thereof) of the neck. The cervical spinal nerves are associated with cervical spinal vertebrae, C1-C7.

In FIG. 16, five example injections areas are illustrated (with dashed ovals). These are provided as illustrative, non-limiting examples of injection sites or regions for the therapy delivery devices described herein. The example injections areas in FIG. 16 are associated with different veins and arteries or portions thereof.

In some such implementations, the injection areas correspond to a stimulation or modulation of a cervical ganglion. The cervical ganglions may be reached by many different routes or vessels, which include internal carotid artery (ICA), external carotid artery (ECA), common carotid artery (CCA), internal jugular vein (IJV), and others, including a combination of the routes.

The internal and external carotid arteries are two branches of the carotid artery, often referred to as the common carotid artery. Access to the cerebral veins and venous sinuses is possible though the internal jugular vein.

Stimulation of one particular cervical ganglion, may indicate or stimulate (e.g., activate or inhibit) one or more other ganglions. For example, stimulation of the inferior ganglion may stimulate the superior cervical ganglion, which may produce a reaction, such as a reduction in vasoconstriction in cerebral vessels. Stimulation may have a singular or combined effect of activation or inhibition of the initial target or downstream connected targets.

A first example injection area 1602 is illustrated at a top left of the diagram and associated with a cervical ganglion, such as the superior cervical ganglion. Access to the first example area may be provided by the internal carotid artery.

A second example injection area 1604 is illustrated at an upper right of the diagram and associated with a cervical ganglion, such as the superior and/or middle cervical ganglions. Access to the second example area may be provided by the external carotid artery.

A third example injection area 1606 is illustrated at a middle of the diagram and associated with a cervical ganglion, such as the superior and/or middle cervical ganglions. Access to the third example area may be provided by the carotid artery, such as in or near a junction of the common carotid artery for internal and external branches thereof.

A fourth example injection area 1608 is illustrated at a central lower portion of the diagram and associated with a cervical ganglion, such as the middle and/or inferior cervical ganglions. Access to the fourth example area may be provided by the carotid artery, such as the common carotid artery.

A fifth example injection area 1610 is illustrated at a bottom left of the diagram and associated with a cervical ganglion, such as the inferior cervical ganglion. Access to the fifth example area may be provided by arteries, such the subclavian artery.

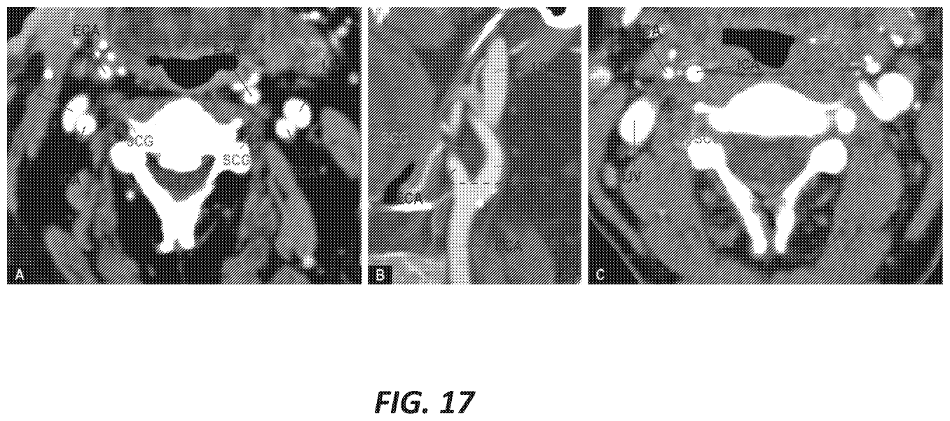

FIG. 17 are pictures of an example of anatomy of a neck of a human. Referring to FIG. 17, representative CTA images of the neck at the level of C3 vertebrae showing SCG location relative to cervical vessels are illustrated. Image (A) illustrates an Axial view. Image (B) illustrates an Oblique sagittal view, and Image (C) illustrates an Axial view of a medially deviated ICA and SCG posterior-lateral to the ICA. The dashed line in Image B denotes the level of common carotid artery bifurcation.

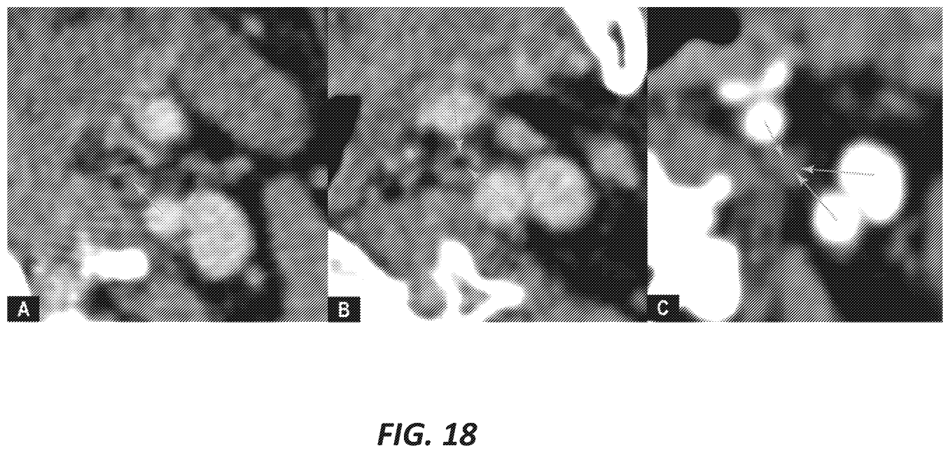

In FIG. 17, arteries and veins proximate the SCG are illustrated as example vessels for potential transvenous treatments for the SCG. However, the layout and anatomy of humans varies from person to person, as shown and described in FIG. 18, and thus, not all possible vessels may have access to the SCG for all patients.

Referring to FIG. 18, representative CTA images of proposed injection paths for different patients are illustrated. In the images of FIG. 18, various viable injections paths are illustrated for each patient, and the differences in the injection paths depicts that access to a ganglion, such as the SCG, may vary from patient to patient.

In the images of FIG. 18, arrows indicated viable injection paths. In Image A, the SCG is accessible from the ICA alone. Injection from ECA is blocked by the vessel branch for the patient of Image A. In Image B, the SCG is accessible from the ICA and the ECA. In Image C, the SCG is accessible from each of the ICA, the ECA, and the IJV.

From the study of location of the SCG for over 159 patients, all SCGs were located along the anterolateral border of longus capitis or longus colli muscle, and 141 out of 159 (88.8%) were located anteromedial or medial to the ICA. Twelve SCGs were located posterolateral (7.55%), and three were posteromedial to the ICA (1.89%). Three SCGs were located anteromedial to ECA (1.89%). A medial deviation of the carotid vessels often co-occurred with a lateralized location of the SCG.

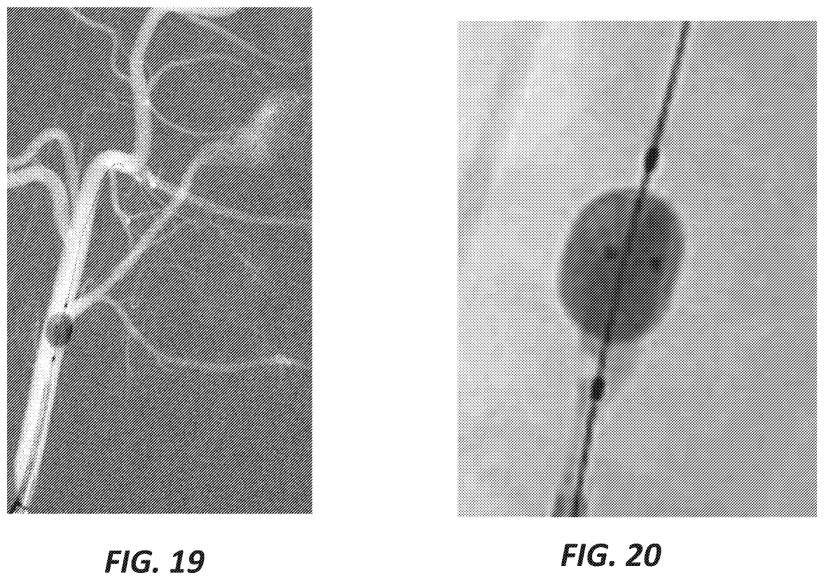

FIG. 19 is an image of catheter applying therapy in a vessel to a ganglion. In FIG. 19, an infusion device a therapy delivery device is positioned in the common carotid artery just below the branch of the ascending pharyngeal artery and external carotid artery. An inflatable device of the infusion device is in an expanded state and the needle has access to the SCG via the wall of the common carotid artery.

FIG. 20 is an image of expanded view of the catheter of FIG. 19. In FIG. 20, multiple radiographic markers are shown. These radiographic markers may help positioning of the therapy device and infusion device thereof. As illustrated in the example of FIG. 20, there are two sets of radiographic markers, such as radio-opaque markings. A first set of markings (e.g., top and bottom as illustrated in FIG. 20, may be used for placement and positioning of the infusion device in the vessel, and a second set of markings may illustrate placement and/or direction of needle puncture for therapeutic intervention.

As part of the present disclosure, specific examples are included below. The examples are for illustrative purposes only and are not intended to be limiting. Those of ordinary skill in the art will readily recognize parameters that can be changed or modified to yield essentially the same results.

### EXAMPLE

### Superior Cervical Ganglion Stimulation Results in Potent Cerebral Vasoconstriction in Swine

Clinical trials were performed on Yorkshire swine to electrically stimulate the superior cervical ganglion to create cerebral hypoperfusion and reduction in blood flow to the brain. By stimulating the superior cervical ganglion to create cerebral hypoperfusion and reduction in blood flow can prove the animal model for one type of cerebral vasospasm. Additionally, the clinical trials included the local administration of lidocaine to the superior cervical ganglion to in part reverse the effects of the electrical stimulation and thereby reduce cerebral hypoperfusion and increase in blood flow to the brain. By inhibiting the stimulation to the superior cervical ganglion to reduce or reverse cerebral hypoperfusion and increase blood flow we can prove the animal model for one treatment of cerebral vasospasm by localized delivery of a local, non-systemic anesthetic.

In a first clinical trial, the following methods were used to evaluate ganglion stimulation and inhibition.

### Methods

### Animal Care.

Use of animals, associated housing/handling and all related experiments were reviewed and approved by the institution's Institutional Animal Care and Use Committee and Animal Research Committee and Division of Laboratory Animal Medicine. All procedure was done in accordance with the Association for Assessment and Accreditation of Laboratory Animal Care International guidelines.

### Anesthesia.

Yorkshire pigs (Sus scrofa) of either gender between 40-50 kg were used. Animals were pre-sedated with intramuscular Telazol and transitioned to inhaled isoflurane for intubation and intravenous (IV) access. General anesthesia was maintained with inhaled isoflurane during the surgical preparation and transitioned to α-chloralose upon completion of the neck dissection to minimize blunting of the autonomic reflexes by isoflurane. A-chloralose was prepared and administered as previously described. All computed tomography images were obtained at least 30 minutes after completely weaning off of the isoflurane.

### Neck Dissection.

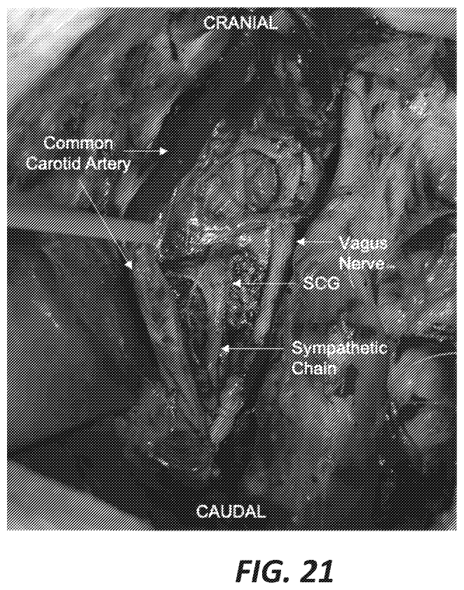

After standard preparation of the surgical field, high anterolateral cervical incisions were made bilaterally along the medial border of the sternocleidomastoideoles and dissection was carried down to the carotid sheath in a typical carotid endarterectomy approach. Contents of the carotid sheath were identified, as illustrated in FIG. 21, while preserving the anatomical integrity of the sympathetic trunk in close proximity to the carotid artery. The superior cervical ganglion was reliably identified near the branch point of the ascending pharyngeal artery.

### SCG Stimulation

Bipolar needles (platinum iridium) were inserted into the identified SCG and was stimulated electrically using a Grass Stimulator. Square wave stimulation began at 1 mA and increased in 0.5 mA increments until ipsilateral mydriasis was noted with fixed supramaximal frequency (10 Hz) and pulse-width (4 ms). SCG was stimulated at twice the magnitude of current required to induce ipsilateral mydriasis to ensure adequate stimulation. Computed tomography perfusion (CTp) scans were obtained at least 30 seconds after the onset of SCG stimulation. SCG stimulation was discontinued after completion of the CT scan, and a minimum of 15 minutes were allotted to ensure all effects of the stimulation had returned to baseline physiologic state, prior to further testing.

### CTp Scan.

Images were obtained using a 2012 Siemens SOMATOM Definition AS Computed Tomography scanner. Bayer HealthCare MEDRAD Stellant contrast injector was used to deliver 50 mL of Omnipaque (iohexol) 300 into the ear vein at a rate of 5 mL/sec with a pressure limit of 325 psi. Imaging acquisition was started before the contrast bolus to capture peak and trough contrast enhancement throughout the study.

### Lidocaine Injection to SCG.

A 29G needle was used to deliver 0.3 mL of 2% Lidocaine HCL to the identified SCG. Electrical stimulation of the SCG was initiated at least 90 seconds after the lidocaine injection to ensure adequate time for the lidocaine to take effect.

### Image Processing and Analysis.

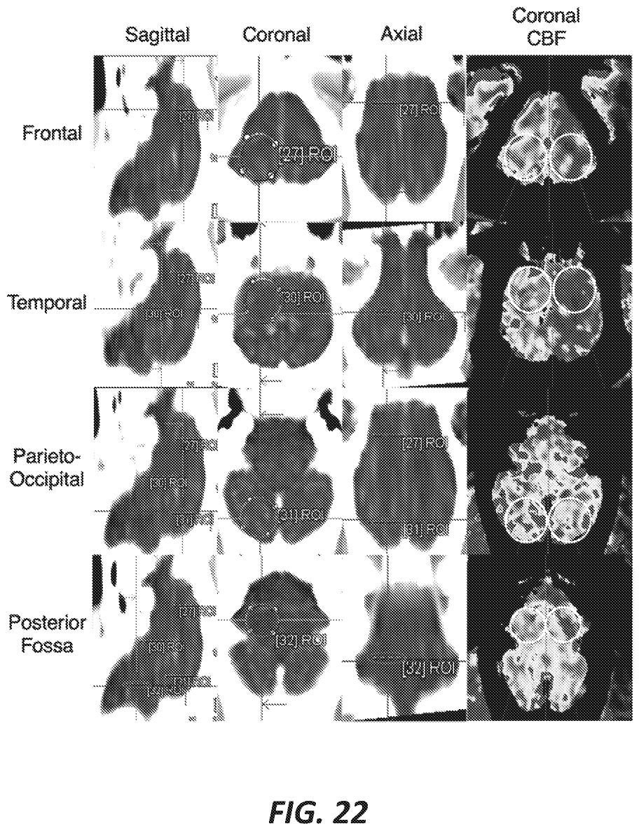

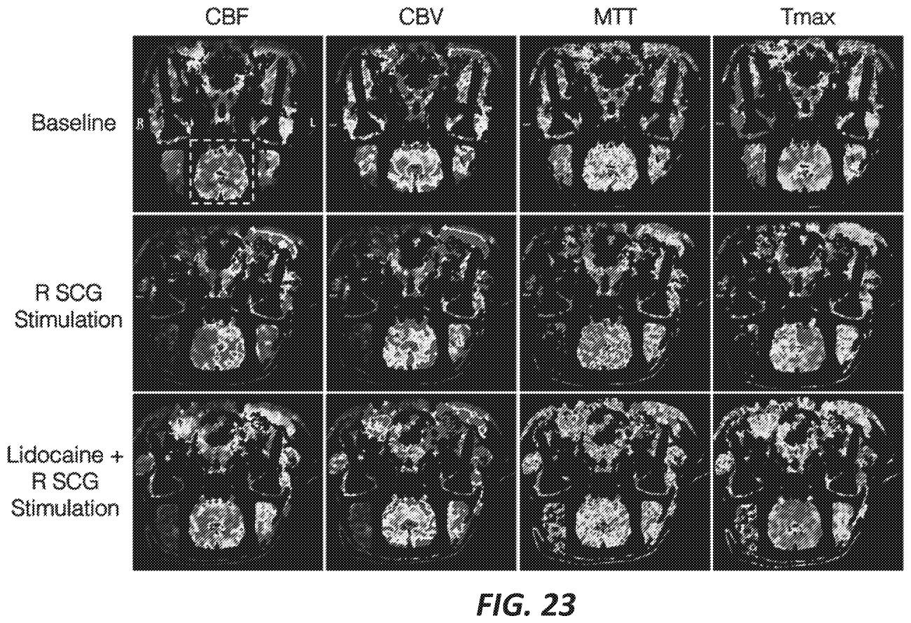

CTp data were analyzed with the commercially available and clinically utilized software, Syngo.via (Siemens Healthcare, Germany). Each scan was evaluated using time-enhancement curves with user-selected references for arterial and venous phases at the common carotid artery and superior sagittal sinus, respectively. Algorithm inherent to the software was used to determine cerebral blood flow (CBF), cerebral blood volume (CBV), mean transit time (MTT) and time-to-maximum (TMax). Regions of interest (ROI) were selected on the right hemisphere at frontal, temporal, parieto-occipital and posterior-fossa regions, as illustrated in FIG. 22. A duplicate corresponding ROI was automatically generated on the left side by the software, by mirroring the right ROI across the mid-sagittal plane, as illustrated in FIG. 23. Mean values for each ROI were calculated and used for subsequent analyses.

### Statistics.

To account for variability between animals and between scans, ROIs described above were analyzed in relation to the laterality of the stimulus (i.e., ipsilateral SCG stimulated ROI as a percentage of contralateral non-SCG stimulated ROI). Two-tailed student's t-test was used on the aforementioned generated values to determine statistical significance between different parameters. The significant level for all tests was set at p=0.05.

### Results

A clinical trial was performed on seventeen Yorkshire swine. Two animals were excluded due to difficulty identifying the SCG and failure to elicit ipsilateral pupillary dilation with electrical stimulation. For each of the Yorkshire swine, CTp data at baseline and with SCG stimulation was obtained during the clinical trial for 29 SCGs (15 left and 14 right stimulations). Additionally, for each of the Yorkshire swine CTp for SCG blockade with stimulation was obtained during the clinical trial for 14 SCGs (8 left and 6 right stimulations).

### SCG Stimulation Causes Ipsilateral Cerebral Perfusion Deficit.

At baseline, it was observed that there was no difference in CBF, CBV, MTT and TMax between the right and left side in the frontal, temporal parieto-occipital and posterior-fossa regions of interest as measured by CTp. Stimulation of the SCG caused ipsilateral global reduction in CBF as measured by CTp in all ROI, as illustrated in FIG. 23.

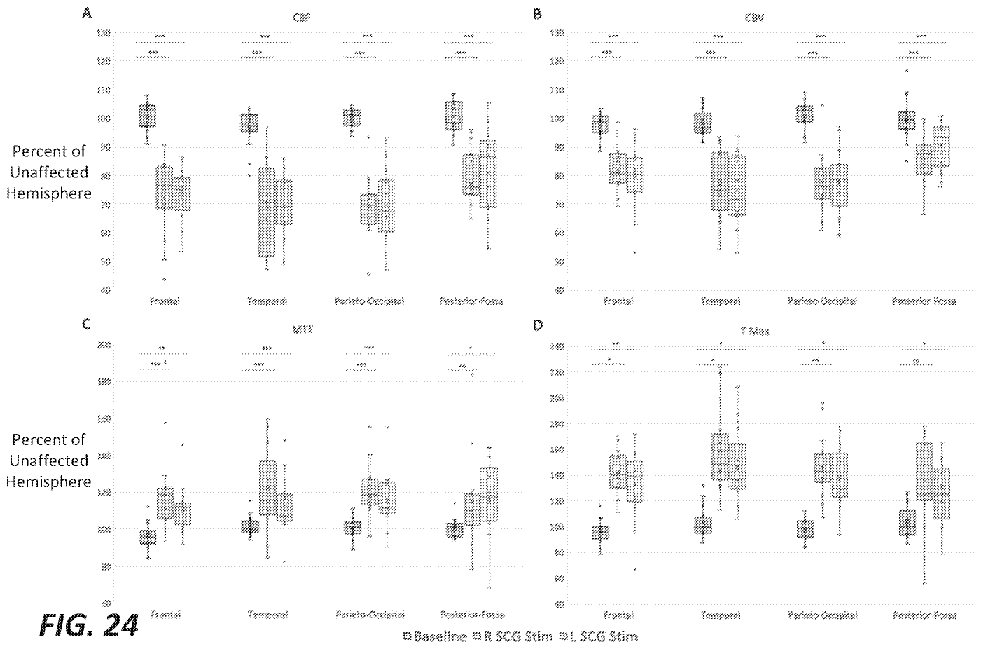

It was also observed during the trials that there was an approximate 20-30% reduction in ipsilateral CBF with SCG stimulation compared to the contralateral non-stimulated side, as illustrated in FIG. 24. Mean CBF decreased by 27.4% and 26.3% in frontal regions (p=4.08×10⁻⁶ and p=1.06×10⁻⁹), 30.8% and 30.4% in temporal regions (p=4.62×10⁻⁵ and p=1.40×10⁻⁷), 30.8% and 30.3% in parieto-occipital regions (p=2.34×10⁻⁷ and p=2.67×10⁻⁷), and 23.0% and 19.0% in posterior-fossa regions (p=2.14×10⁻⁴ and p=2.85×10⁻⁴) with right and left side SCG stimulations respectively, compared to non-stimulated contralateral side.

In addition, CBV also decreased by approximately 10-25% with ipsilateral SCG stimulation, as illustrated in FIG. 24. Mean CBV decreased by 17.8% and 20.6% in frontal regions (p=6.93×10⁻⁶ and p=2.27×10⁻⁵), 23.0% and 25.1% in temporal regions (p=4.45×10⁻⁵ and p=3.25×10⁻⁶), 22.0% and 22.2% in parieto-occipital regions (p=3.94×10⁻⁶ and p=1.30×10⁻⁶), and 14.0% and 9.69% in posterior-fossa regions (p=2.86×10⁻⁴ and p=2.64×10⁻³) with right and left sided SCG stimulations respectively.

Although, both MTT and TMax increased with ipsilateral SCG stimulation, consistent with perfusion deficit, there was greater variability in the mean change, as illustrated in FIG. 24. Mean MTT increased by 21.9% and 10.7% in frontal regions (p=4.16×10⁻³ and p=1.03×10⁻³), 21.6% and 13.0% in temporal regions (p=0.010 and p=0.047), and 21.7% and 15.7% in parieto-occipital regions (p=2.09×10⁴ and p=2.24×10⁻³) with right and left sided SCG stimulation respectively. While left SCG stimulation caused a 17.9% increase in MTT posterior-fossa regions that was statistically significant (p=7.16×10⁻³), right SCG stimulation caused a 15.6% increase in MTT that was approaching significance (p=0.068). Mean Tmax increased by 42.4% and 33.3% in frontal region (p=1.23×10⁻⁷ and p=1.45×10⁻⁴), 59.1% and 46.7% in the temporal region (p=3.85×10⁻⁵ and 4.91×10⁻⁵), 46.3% and 38.2% in the parieto-occipital region (p=1.51×10⁻⁵ and p=9.34×10⁻⁶), and 35.8% and 25.5% in posterior-fossa region (p=7.07×10⁻³ and p=5.81×10⁻³) with right and left sided SCG stimulation respectively.

### Prior SCG Blockade Prevents Cerebral Hypoperfusion in Setting of SCG Stimulation.

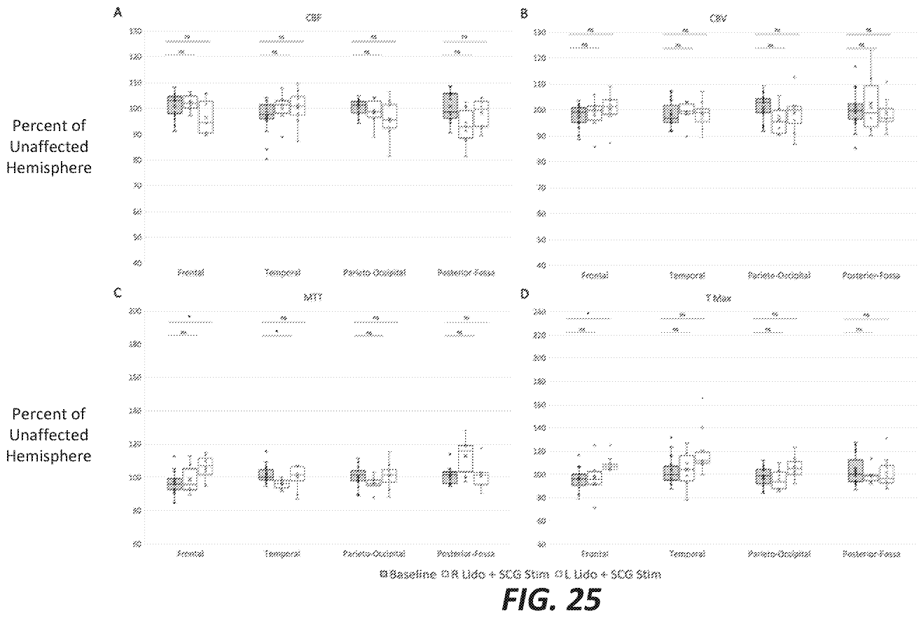

Prior lidocaine administration to the SCG inhibited the effects of SCG stimulation described above and restored cerebral perfusion. Mean CBF was less than 10% different from all measured regions compared to contralateral non-stimulated regions and this difference was not statistically significant compared to baseline (all p>0.10), as illustrated in FIG. 25. Similarly mean CBV was less than 5% different from all measured regions compared to contralateral non-stimulated regions and this difference was not statistically significant compared to baseline (all p>0.10).

Again, there was more variability with MTT and Tmax parameters when lidocaine was administered prior to SCG stimulation. Apart from the left frontal region, which had a mean 5.82% increase in MTT compared to the non-stimulated right frontal region (p=0.0144), and the right temporal region, which had a mean 4.13% decrease in MTT compared to the non-stimulated left temporal region (p=8.06×10⁻³), all other measured MTT means were within 15% of the non-stimulated contralateral side without statistical difference. For mean Tmax, measured values were within 20% of the non-stimulated contralateral side without statistical difference, except for the left frontal region that showed an 8.80% increase in mean Tmax compared to the non-stimulated right frontal region (p=2.10×10⁻³).

### Discussion

To our knowledge, we are the first group to demonstrate true cerebral hypoperfusion from direct activation of the sympathetic nervous system in a large mammal. We used Yorkshire swine to develop a large mammal model of sympathetically-mediated vasospasm for translation into clinical application. Large mammals, such as the swine, more closely resemble human anatomy without the cost and other restrictions associated with canine and primate models. Additionally, swine cardiovascular anatomy is similar to that of humans and prior studies have used swine as experimental models to study cerebral vasospasm.

An essential component to our model was the use of α-chloralose. α-chloralose is the preferred anesthetic for animal studies evaluating autonomic function as it provides depth of anesthesia, while maintaining high level of basal autonomic tone that is easily modified to produce robust responses.

While the superior cervical ganglion is recognized to have sympathetic efferents, information regarding synaptic coverage, number of synapses and variability dependent on animal size and weight have not been well-characterized. Hence, although we used ipsilateral mydriasis as a means to confirm sympathetic activation, we decided to stimulate the SCG at twice the current required to achieve mydriasis in order to ensure we elicited a robust activation of the SCG.

In addition to cerebral hypoperfusion, SCG stimulation induced a more striking perfusion deficit in extra-cranial regions evident on the CTp scans. This change was most evident in the snout, where there was blanching of the skin on the side of SCG stimulation. Although the carotid system in animals have greater proportional contribution to the external carotid distribution than seen in humans to supply the larger facial and masticatory muscles, studies have shown anastomoses between external and internal carotid arteries in the swine that contribute to cerebral blood supply. Therefore, we cautiously speculate that sympathetically-mediated hypoperfusion may have a greater effect on cerebral perfusion in humans than shown in our swine model, as the swine brain is more protected by significant extra-cranial collateral flow. Despite this added “protection,” we were able to demonstrate significant cerebral perfusion deficits.

With respect to SCG blockade, we decided against using a longer-acting agent (i.e. Marcaine, acetylcholine receptor inhibitors), as doing so would increase the time required to recover to baseline and thereby unnecessarily lengthen the overall anesthesia time. In our study, we used a readily-available, short-acting sodium channel blocker, lidocaine. Although the exact duration of this effect was not measured, we found that we were able to re-demonstrate SCG-mediated ipsilateral perfusion deficit approximately 30 minutes after lidocaine administration (data not shown). Additionally, we did not see an increase in ipsilateral cerebral perfusion with lidocaine administration alone (in the absence of SCG stimulation) (data not shown). This suggests either low baseline sympathetic tone or a robust baseline brain perfusion that exceeds any hyper-perfusion attempts made with SCG inhibition alone.

The role of parasympathetic activity in cerebral perfusion is still under investigation. While Yarnitsky et al have shown dilation of cerebral vessels with parasympathetic stimulation in a subarachnoid hemorrhage induced model of vasospasm in dogs, it is unclear whether sympathetic hyperactivity or parasympathetic underactivity has a stronger influence on cerebral vasospasm. Regardless, the ability to modulate both sympathetic and parasympathetic nervous systems remains a promising potential therapeutic intervention for cerebral vasospasm and deserves further investigation.

The main limitation to our study is perhaps, the simplification of using sympathetically-mediated hypoperfusion as a model for cerebral vasospasm. Recent studies show strong sympathetic activity in subarachnoid hemorrhage induced vasospasm in numerous animal models and suggest sympathetic modulation as a promising therapy. We recognize that true cerebral vasospasm involves a complex interplay between various factors, but ultimately the clinically relevant end result of hypoperfusion is what we were able to achieve in our model.

Another limitation arises from the differences in swine and human cerebrovascular anatomy. Unlike in humans, swine common carotid artery bifurcates into the external carotid artery and the ascending pharyngeal artery, which later forms a rete mirabile before giving rise to internal cerebral arteries. The rete mirabile, an arterial meshwork, is thought to have and evolutionary role in maintaining cerebral blood flow and blood temperature regulation in many mammalian species. As such, the presence of carotid rete mirabile in swine may help guard against cerebral perfusion deficit. It is our belief that the observed perfusion deficit would have been even more pronounced without the protection of the rete mirabile and the extensive external to internal collaterals. While this is a limitation of the model, it may imply that the results are even more translatable to humans, who do not have such pronounced vascular protection mechanisms.

Some have challenged the notion that vasospasm is the primary causative mechanism for distal cerebral ischemia (DCI) following subarachnoid hemorrhage, endorsing spreading depolarization, microcirculatory dysfunction, disrupted cerebral autoregulation, and early brain injury as separate contributing factors. While the exact pathophysiology of DCI in this setting remains unclear, resolution of vasospasm continues to be associated with good neurologic outcomes in patients following subarachnoid hemorrhage. Accordingly, reversal of cerebral vasospasm remains the standard of care and discovery of new methods for reversing vasospasm continues to be an area of interest in the cerebrovascular research community

### Conclusion

Our first clinical study demonstrates that sympathetic hyperactivity can lead to true cerebral hypoperfusion in a large mammal swine model. Furthermore, this activity can be inhibited with SCG blockade to restore cerebral perfusion. Accordingly, we propose further investigations into inhibition of SCG as a potential therapeutic option for cerebral vasospasm

A second clinical trial was also performed to further investigations into inhibition of SCG as a potential therapeutic option for cerebral vasospasm. A brief abstract and introduction of the second trial is provided below before the details of the second clinical trial.

From the first trial, sympathetic activity from the superior cervical ganglion (SCG) has been shown to cause cerebral hypoperfusion in swine, similar to that seen with clinical cerebral vasospasm. Although the mechanism of such perfusion deficit has been speculated to be from pathologic cerebral vasoconstriction, the extent of sympathetic contribution to vasoconstriction has not been well-established.

As an objective of the second trial, we aimed to demonstrate that SCG stimulation in swine leads to significant cerebral vasoconstriction on angiography. Additionally, we aimed to show that inhibition of SCG can mitigate the effects of sympathetic-mediated cerebral vasoconstriction.

In the second clinical trial, the methods included surgically identifying five SCGs in Yorkshire swine. The five SCG were electrically stimulated to achieve sympathetic activation. Cerebral angiography was performed to measure and compare changes in cerebral vessel diameter. Syngo iFlow was also used to quantify changes in contrast flow through the cerebral and neck vessels.

During the second clinical trial we observed that SCG stimulation resulted in 35-45% narrowing of the ipsilateral ascending pharyngeal, anterior middle cerebral and anterior cerebral arteries. SCG stimulation also decreased contrast flow through ipsilateral ascending pharyngeal, internal carotid and anterior cerebral arteries as seen on iFLow. These effects were prevented with prior SCG blockade. Minimal changes were seen in the posterior cerebral, posterior middle cerebral and internal carotid arteries with SCG stimulation.

From the second clinical trial we concluded that SCG stimulation results in significant luminal narrowing and reduction in flow through intracranial arteries in swine. The results of sympathetic hyperactivity from the SCG closely models cerebral vasoconstriction seen in human cerebral vasospasm. SCG inhibition is a potential promising therapeutic approach to treating cerebral vasospasm. The full study is provided below

### Introduction

Cerebral vasospasm is a disease of pathologic narrowing of the cerebral vasculature leading to diminished cerebral blood flow, resulting in significant morbidity and stroke. Various modalities are used to assess for cerebral vasospasm, including clinical exam, elevated Lindegaard ratio on transcranial doppler flow velocity measurements, or visualizing irregular vessels on axial imaging, such as computed tomography angiography (CTA). However, the gold standard method for diagnosing cerebral vasospasm is the observation of irregularly diminished cerebral vessel caliber on a digital subtraction angiography. Furthermore, the severity of vasospasm is determined using a widely accepted grading scale based on the degree of cerebral vessel luminal narrowing.

Although the exact mechanism of cerebral vasospasm remains unclear, sympathetic hyperactivity has been shown to be a significant contributor. Sympathetic innervation to the ipsilateral cerebral vasculature originates from the superior cervical ganglion (SCG). We have previously shown in the first clinical trial that, in a swine model, electrical stimulation of the SCG results in significant cerebral perfusion deficit. However, a purely sympathetic mediated cerebral vasoconstriction has not been characterized in a large mammal model on angiography.

In this study, we aimed to show that sympathetic activation of the SCG is sufficient to induce true cerebral vasoconstriction. Furthermore, we demonstrate that prior pharmacologic blockade of SCG inhibits this sympathetic-mediated cerebral vasoconstriction.

### Methods

### Animal Care.

Use of animals, associated housing/handling and all related experiments were reviewed and approved by the institution's Institutional Animal Care and Use Committee and Animal Research Committee and Division of Laboratory Animal Medicine. All procedures were conducted in accordance with the Association for Assessment and Accreditation of Laboratory Animal Care International guidelines.

### Anesthesia/Neck Dissection/SCG Stimulation/SCG Blockade.

Yorkshire pigs (Sus scrofa) of either gender between 40-50 kg were used. Animals were anesthetized and bilateral carotid sheath contents were surgically exposed as previously described*. Anesthesia was transitioned to alpha-chloralose and the SCG were stimulated as previously described. An SCG was stimulated for 30 seconds prior to angiography imaging. For SCG blockade, a 29G needle was used to deliver 0.3 mL of 2% Lidocaine HCL to the identified SCG. Ninety seconds were allotted after lidocaine administration prior to SCG stimulation, with subsequent angiography done 30 seconds after stimulation.

### Digital Subtraction Angiography (DSA).

Diagnostic angiography procedures were performed using a Siemens Artis Zeego C-Arm. Through a femoral arterial sheath, a 5F diagnostic catheter was introduced and navigated over a glidewire superiorly. The ascending pharyngeal artery was catheterized under roadmap guidance. DSA was performed in a standardized fashion using a Medrad Mark V ProVis injector to deliver 6 mL of Omnipaque (iohexol) 300 through the 5F catheter, with a flow rate of 3 mL/s and maximum pressure of 300 psi. All images were obtained at the same magnification and view for each experiment set.

All obtained images and series were transferred to, and stored in the institution's picture archiving and communication system (PACS).

### Vessel Measurements.

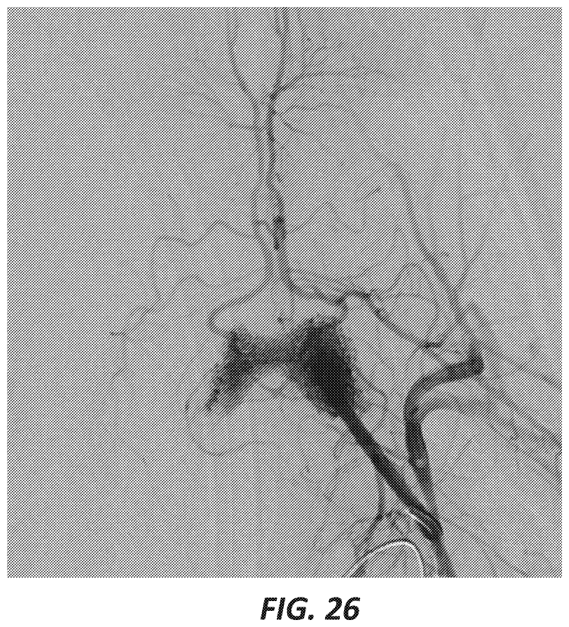

Vessel diameters were measured using the institution's PACS system. Location of the vessel diameter measurements for various arteries are as shown in FIG. 26. Diameters of ascending pharyngeal artery (APA), anterior cerebral artery (ACA), anterior middle cerebral artery (aMCA), posterior middle cerebral artery (pMCA), internal carotid artery (ICA) and posterior cerebral artery (PCA) were measured. All measurements were done under four times the original magnification to ensure accurate measurements of vessel diameter.

### Contrast Flow Measurements.

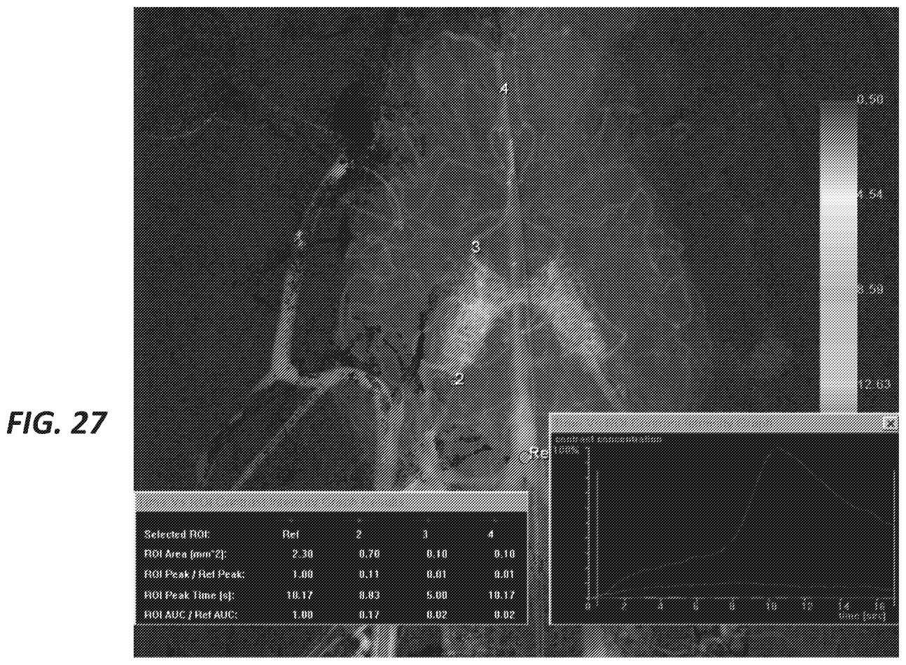

Angiography series were further analyzed with the commercially available and clinically utilized software, Syngo iFlow (Siemens Healthcare, Germany). Using the algorithm inherent to the software, a colorimetric map was generated to visualize time of maximum contrast intensity detection at various regions, as illustrated in FIG. 27. Regions of interest (ROI) were selected along the APA, ICA and ACA, where vessel diameters were measured. Given the small calibers of MCA and PCA vessels, they were not reliably identified on the colorimetric maps and therefore, were excluded in these measurements. Contrast flow metrics were calculated and used as a surrogate for blood volume flow. ROIs were used to plot percent contrast intensity detected over time. Area under this generated curve was then calculated and used as a measure of total contrast flow through the vessel over the defined time interval, as illustrated in FIG. 27. Location and area of ROI was kept identical between different angiograms to ensure consistency.

The contrast flow in each ROI was normalized to contrast flow passing through the confluence of sinus (torcula) as the reference point. The torcula was chosen as the reference point because it was determined to be the vessel that consistently has maximum contrast flow, and is not affected by SCG stimulation, thereby comparable between angiograms. Using a reference point just distal to the catheter tip, for example would introduce variability depending on degree of vasoconstriction of the ascending pharyngeal artery and therefore not selected.

### Statistics.

To account for normal variation in vessel diameter between animals and laterality, vessel diameters were represented as a percent of the diameters measured at baseline (no SCG stimulation) and compared between groups. Two-tailed student's t-test was used to determine statistical significance between groups. The significant level for all tests was set at p=0.05.

### Results

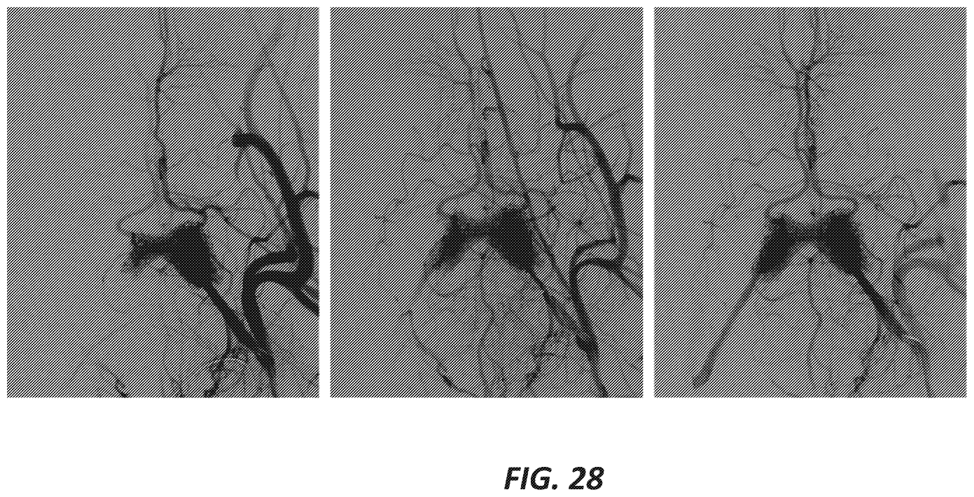

Five SCGs, from 3 animals, were used in our study. One SCG was excluded from data analysis secondary to difficulty identifying the ganglion and failure to demonstrate ipsilateral mydriasis with baseline stimulation. There was significant vasoconstriction seen in both intracranial and extracranial arteries with SCG stimulation, as illustrated in FIG. 28.

SCG Stimulation Causes Vasoconstriction in APA, aMCA and ACA.

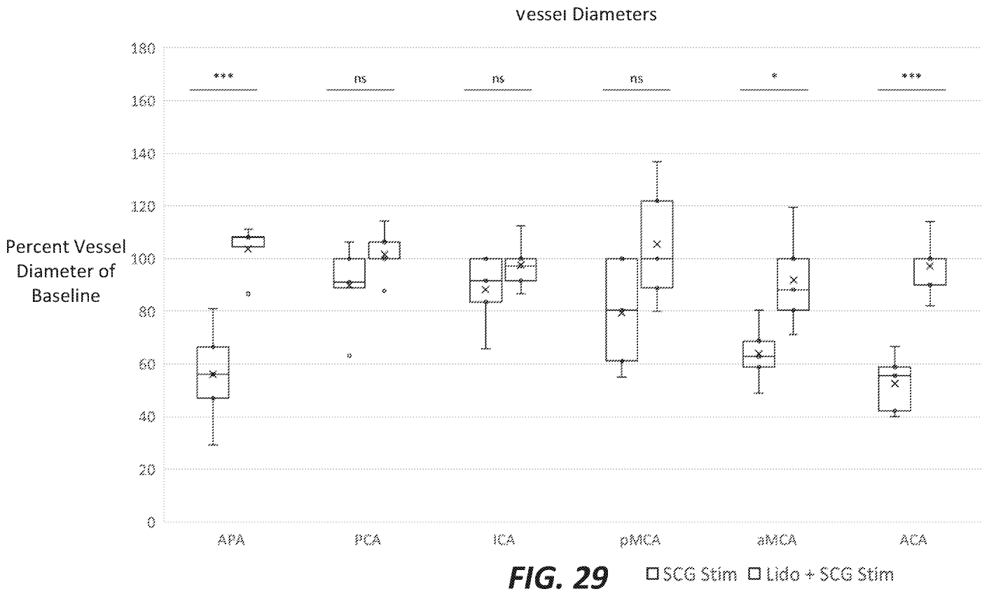

SCG stimulation resulted in significant vasoconstriction of APA, aMCA and ACA, as illustrated in FIG. 29. Mean vessel diameter decreased by 44% in APA (p=0.011), 36.1% in aMCA (p=3.89×10−3), and 47.3% in ACA (p=2.46×10−3), with ipsilateral SCG stimulation compared to baseline. While other vessel diameters decreased with SCG stimulation, they did not meet statistical significance. When SCG was inhibited with lidocaine prior to SCG stimulation, there was no significant difference in vessel diameter compared to baseline (all p-values>0.2). Similarly, mean vessel diameters were statistically smaller in APA, aMCA and ACA with SCG stimulation, compared to when SCG was inhibited with lidocaine prior to stimulation, as illustrated in FIG. 29.

### SCG Stimulation Decreases Contrast Flow in APA, ICA and ACA.

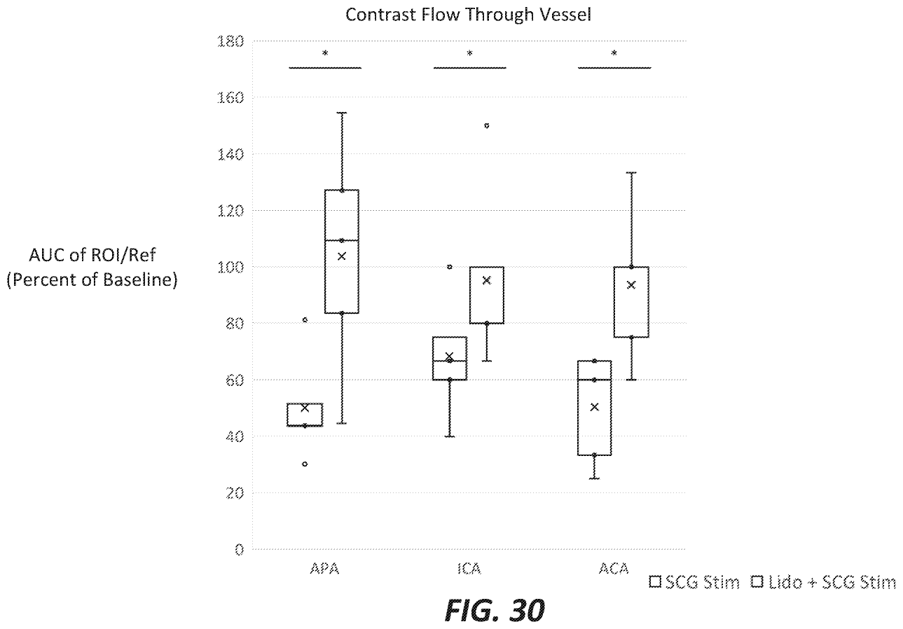

SCG stimulation significantly reduced the contrast flow through APA, ICA and ACA, as illustrated in FIG. 30. Mean contrast flow decreased by 49.4% in APA (p=0.038), 31.7% in ICA (p=0.035) and 49.7% in ACA (p=8.58×10−3) with ipsilateral SCG stimulation compared to baseline. When SCG was inhibited with lidocaine prior to SCG stimulation, there was no significant difference in contrast flow through the APA, ICA and ACA compared to baseline (all p-values>0.3). Again, mean contrast flow through the vessels were significantly reduced with SCG stimulation, compared to when SCG was inhibited prior to stimulation, as illustrated in FIG. 30.

### Discussion

To the authors' knowledge, this is the first study to demonstrate with DSA that sympathetic activation is sufficient to induce potent cerebral vasoconstriction in a large mammal vasospasm model. The SCG electrical stimulation procedure outlined above shows promise as a novel and easily reversible large-mammal animal model for cerebral vasospasm without the challenges associated with models established by autologous blood injections to the subarachnoid space, such as higher acuity of animal care and repeated DSA monitoring. Degree of vasospasm in swine vasospasm models have been traditionally assessed using diametric measurements, with ICA caliber narrowing ranging from 16-34% in autologous blood injection models. In our study, ICA caliber reduced by an average of ˜10% from baseline, with an accompanying 31.7% reduction in ICA perfusion comparable to findings from other swine SAH experiments. While further studies are warranted to better establish our protocol as a model for SAH-induced vasospasm, our results, in conjunction with our prior work showing cerebral perfusion deficits seen in SCG stimulation, offer a compelling, sympathetic-driven model of cerebral vasospasm in large mammals.

Our prior study demonstrated that SCG stimulation results in a holo-hemispheric cerebral perfusion deficit in swine. However, this study demonstrates in a more granular fashion that only some of the cerebral arteries had significant vasoconstrictive response to SCG stimulation. This discrepancy between greater perfusion deficit and more limited individual vasoconstrictive changes, may be due to significant vasoconstriction of the ascending pharyngeal artery. While there exists an artery named the “internal carotid artery” in swine, its anatomic location and orientation is vastly different from that of humans'. In fact, some argue that the ascending pharyngeal artery in the swine is a more similar structure to the human ICA, as they both branch off the common carotid artery and act as the main arterial feeder to intracranial structures. Hence, while some vessel calibers seem unaffected by SCG stimulation, the reduction in cerebral blood flow from narrowing of the ascending pharyngeal artery in swine may be sufficient to cause perfusion deficits. This is further supported by our observation of a significant reduction in contrast flow through the ICA, as illustrated in FIG. 30, despite overall unchanged vessel diameter, with SCG stimulation.

Another consideration for the variability in cerebral vasoconstriction seen with SCG stimulation may be due to variability in our ability to accurately identify and stimulate the entire SCG. We have seen in our prior study that we were able to induce ipsilateral mydriasis and ipsilateral cerebral perfusion deficit with stimulation of the sympathetic chain above or below the SCG, albeit usually with higher current. Partial stimulation of the SCG might lead to variations in cerebral vasoconstriction based on the map of the SCG as it relates to the downstream vascular targets. Furthermore, while recent studies have shown differential regulation of anterior and posterior cerebral circulation in humans, there may be additional autonomic innervation of cerebral vessels in swine that remains yet to be elucidated. This may explain the lack of significant changes in vessel caliber for PCA and even pMCA vessels with SCG stimulation.

Similar to our prior work that showed greater CT perfusion deficit in the extracranial distribution, we observed a more significant vasoconstrictive effect on extracranial than intracranial vessels with SCG stimulation. As previously mentioned, swine have greater emphasis on extracranial vasculature compared to humans, to supply larger facial muscles and the snout. Accordingly, we suspect that swine have greater number of sympathetic efferents to the extracranial than intracranial vessels. This could also explain the potent vasoconstriction response of the ascending pharyngeal artery. It is important to recognize that in swine, many extracranial arteries have been found to form anastomoses with intracranial arteries to contribute to the cerebral blood supply. Given the significant vasoconstriction of the extracranial circulation in swine, our model may underestimate the role of SCG in altering intracranial vessel caliber in humans.

There is debate in the literature regarding the association between cerebral vasospasm and delayed cerebral infarction. Traditionally, cerebral ischemia and poor patient outcomes following subarachnoid hemorrhage have been attributed to arterial narrowing and cerebral vasospasm. However, it has been proposed vasospasm may not be the main cause of delayed cerebral ischemia (DCI), and that alternative disease processes such as global ischemia, surrounding inflammation and cortical spreading depolarization may be more closely associated. Regardless of which specific phenomenon is responsible for DCI, sympathetic regulation of cerebral vasculature remains an important topic and potential therapeutic option for improving patient outcomes.

A newly emerging disease process that is relevant to our study is reversible cerebral vasoconstriction syndrome (RCVS). The term RCVS was coined by Calabrese et al in 2007 to encompass various cerebral vasoconstriction syndromes described since the 1970s. RCVS is thought to be more frequent than previously predicted and has been associated with cerebral ischemia, edema, stroke and even non-aneurysmal subarachnoid hemorrhage. Recent studies have shown that the pathophysiologic mechanism of RCVS may be due to sympathetic overactivity. The findings in our study support this notion, as sympathetic activation clearly resulted in reversible cerebral vasoconstriction in a large mammal model. In this sense, our findings suggest that pharmacologic SCG blockade may be an effective treatment for patients suffering from RCVS in addition to cerebral vasospasm.

Limitations of our study include the small sample size. However, given the striking and obvious changes in vessel caliber seen with SCG stimulation, we were able to demonstrate statistical significance with this sample size. Our study may have been underpowered to detect changes in vessel diameters for other vessels, including PCA, pMCA and ICA. Additionally, we were unable to accurately determine changes in contrast flow in MCA and PCA vessels using the Syngo iFlow system as mentioned above. Further studies with higher magnification may have been used to better detect such changes in these smaller vessels. However, the small size of the vessels and the thickness of the swine cranium may limit proper visualization to detect these changes.

### Conclusion

Our study demonstrates that sympathetic activation through SCG stimulation can induce significant vasoconstriction of intracranial vessels and reduction of blood flow in a large mammal swine model. In addition, SCG blockade is able to mitigate this effect and prevent this vasoconstriction. Sympathetic mediation through SCG inhibition is a potential promising therapeutic intervention for diseases involving pathologic cerebral vasoconstriction.

An additional study was conducted regarding the safety of puncturing such large and sensitive vessels of the neck and head. In the study, a common carotid artery was punctured ten times by a needle of a catheter based therapy device. After puncturing the carotid artery ten times, the swine was monitored by MRI and digital subtraction angiography (DSA). The MRI indicated no evidence of stroke in the brain or excessive bleeding at the puncture site. DSA indicated no evidence of vessel injury/dissection or active extravasation of contrast dye/blood.

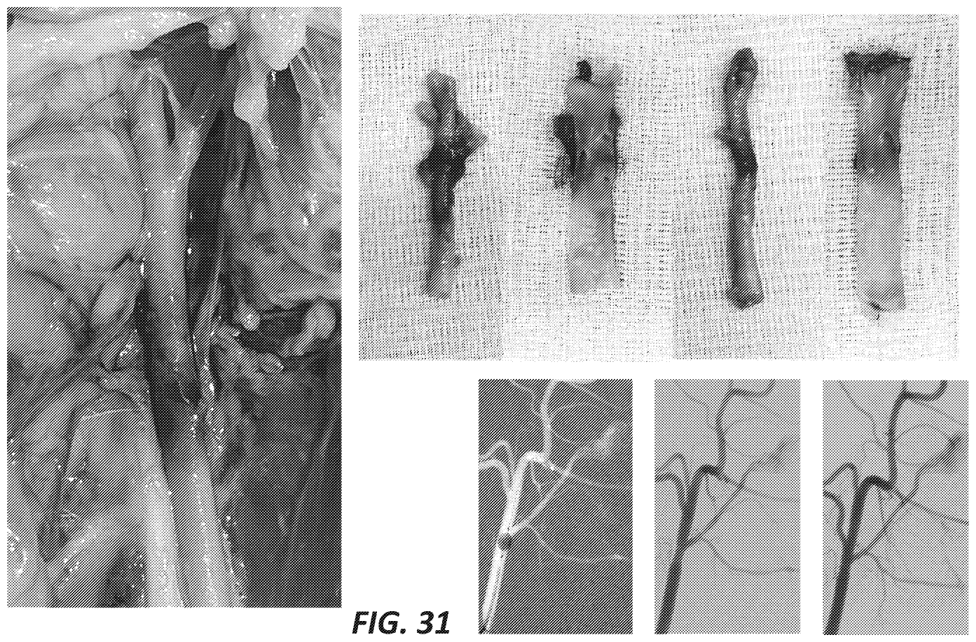

FIG. 31 illustrates multiple photographs taken during the study of repeated puncturing of the carotid artery. The photographs show the carotid artery after puncturing the carotid artery during operation, after removal of the device and before harvesting, and after harvesting and dissection. FIG. 31 (bottom right) depicts multiple images of a carotid artery during a procedure with repeated puncturing. FIG. 31 (left) depicts an image of a carotid artery after repeated puncturing and before harvesting. FIG. 31 (top) depicts multiple images after harvesting and dissection.

During the procedure, the MRI and DSA images show only a temporary narrowing of the carotid artery shortly after puncture, consistent with local, temporary vasospasm. After a couple of minutes, the carotid artery returned to normal size and no excessive contrast or bleeding was visible outside of the carotid artery due to the repeated puncture.

After the procedure and removal of the catheter, the carotid artery, which was punctured repeatedly, was harvested and dissected. During harvesting, there was evidence of only minimal prior bleeding from the repeated punctures as only light adventitial staining was present in the location of the punctures. Upon dissection of the carotid artery after harvesting, the wall of the carotid artery was structurally intact and showed no signs of structural or collateral damage. Accordingly, it was found that such larger and sensitive vessels, such as cervical, cerebral, or coronary, could safely withstand transvascular therapy application in contrast to prior knowledge.

The above specification and examples provide a complete description of the structure and use of illustrative implementations. Although certain implementations have been described above with a certain degree of particularity, or with reference to one or more individual implementations, those skilled in the art could make numerous alterations to the disclosed implementations without departing from the scope of this invention. As such, the various illustrative implementations of the methods and systems are not intended to be limited to the particular forms disclosed. Rather, they include all modifications and alternatives falling within the scope of the claims, and implementations other than the one shown may include some or all of the features of the depicted implementation. For example, elements may be omitted or combined as a unitary structure, or connections may be substituted. Further, where appropriate, aspects of any of the examples described above may be combined with aspects of any of the other examples described to form further examples having comparable or different properties or functions, and addressing the same or different problems. Similarly, it will be understood that the benefits and advantages described above may relate to one implementation or may relate to several implementations.

The claims are not intended to include, and should not be interpreted to include, means-plus- or step-plus-function limitations, unless such a limitation is explicitly recited in a given claim using the phrase(s) “means for” or “step for,” respectively.

1. A method for stimulating nervous system tissue using an endovascular transvascular technique, the method comprising:
inserting a therapy delivery device comprising an infusion device into a vessel of a patient;
positioning the therapy delivery device in the vessel near nervous system tissue; and
applying, from the infusion device, a therapeutic across a wall of the vessel into the nervous system tissue of the patient to treat the nervous system tissue and a neurologic condition.

2. The method of claim 1, wherein applying the therapeutic across the wall of the vessel to inhibit or activate the nervous system tissue includes:
injecting a medication across the wall of an artery or vein, wherein the medication is an amino amide type local anesthetic, an ester group base local anesthetic, lidocaine, a cancer medication, or configured to reduce luminal narrowing and cause an increase in blood flow through intracranial arteries.

3. (canceled)

4. (canceled)

5. (canceled)

6. The method of claim 1, wherein applying the therapeutic across the wall of the vessel to inhibit or activate the nervous system tissue includes:
injecting a biologic across the wall of an artery or vein.

7. The method of claim 1, wherein the therapeutic is injected into nervous system tissue of a ganglion or injected into cerebral spinal fluid spaces.

8. The method of claim 1, wherein the therapeutic is injected into tissue of a superior cervical ganglion (SCG) and configured to inhibit the SCG.

9. (canceled)

10. (canceled)

11. The method of claim 1, wherein positioning the therapy delivery device in the vessel near the nervous system tissue includes:
advancing the therapy delivery device in an artery or vein to a point adjacent a cervical ganglion of a sympathetic nervous system (SNS), such that, upon activation, the therapy delivery device injects the therapeutic into or across the wall of the artery or vein, wherein the artery or vein is a carotid artery or a branch thereof, a cerebral artery or a branch thereof, a vertebral artery or a branch thereof, an internal jugular vein, an external jugular vein, a cranial venous sinus, a cerebral vein, or ascending pharyngeal artery.

12. (canceled)

13. (canceled)

14. (canceled)

15. The method of claim 1, wherein the therapeutic is configured to target sympathetic fibers and decrease cerebral perfusion and cerebral blood flow, or wherein the vessel is a subclavian artery and the therapeutic is configured to modulate nerves of a brachial plexus.

16. (canceled)

17. The method of claim 1, wherein the method further comprises:
applying, from an electrical stimulation device of the therapy delivery device, electrical stimulation therapy to the wall of the vessel or across the wall of the vessel into the nervous system tissue to modulate the nervous system tissue; or
aspirating, by an aspiration device of the therapy delivery device, a bodily fluid across the wall of the vessel.

18. The method of claim 1, further comprising, after positioning the therapy delivery device:
inflating a balloon, wherein inflation of the balloon is configured to secure the therapy delivery device in place, and wherein inflation of the balloon is further configured to cause actuation of the infusion device and application of the therapeutic across the wall of the vessel, cause obstruction of blood flow, or both.

19. (canceled)

20. (canceled)

21. The method of claim 1, further comprising:
applying, therapy delivery device, a second therapy to or across the wall of the vessel to inhibit the nervous system tissue to treat the neurologic condition.

22. The method of claim 1, wherein treating the nervous system tissue includes stimulating, activating, modulating, or inhibiting the nervous system tissue or adjacent tissue which affects the nervous system tissue, and wherein the neurologic condition is cerebral vasospasm.

23. (canceled)

24. (canceled)

25. (canceled)

26. A method for applying therapy using an endovascular transvascular technique, the method comprising:
receiving, by a therapy delivery device while in a vessel of a patient, a therapeutic, the therapy delivery device comprising an infusion device; and
providing, by the infusion device, the therapeutic across a wall of the vessel into tissue of the patient.

27. (canceled)

28. A therapy delivery device, the therapy delivery device comprising:
a catheter having a distal end and a proximal end; and
an infusion device coupled to the proximal end of the catheter, the infusion device configured to inject an agent into or across a wall of a vessel.

29. The therapy delivery device of claim 28, wherein the infusion device is a microneedle, is a guideable or steerable needle configured to be advanced within a lumen of the catheter, or is an expandable stent including a retractable puncturing device.

30. (canceled)

31. The therapy delivery device of claim 28, wherein the infusion device includes a needle and an inflation device, the needle coupled to the inflation device and configured to be inserted into or through the wall by expansion of the inflation device.

32. The therapy delivery device of claim 31, wherein the inflation device is a single balloon, and wherein the single balloon is configured to restrain the catheter in position and apply the infusion device, wherein the single balloon has a torus shape including a central aperture configured to enable annular flow.

33. (canceled)

34. The therapy delivery device of claim 31, wherein the inflation device is a dual modulus balloon.

35. The therapy delivery device of claim 31, wherein the inflation device includes multiple balloons, and wherein the multiple balloons include multiple injection balloons, and wherein each of the multiple injection balloons are configured to apply therapy either into the vessel or across the vessel.

36. (canceled)

37. (canceled)

38. (canceled)

39. (canceled)

40. The therapy delivery device of claim 28, further comprising:
a contrast delivery device;
an aspiration device;
an electrical stimulation device configured to apply electrical stimulation therapy; or
any combination thereof.

41 The therapy delivery device of claim 28, wherein the catheter includes a sheath comprising a hydrophilic coating and coiled wire supports or struts, wherein the distal end of the catheter has a rounded tip, and wherein the distal end of the catheter has a first stiffness different from a second stiffness of the proximal end of the catheter.

42. (canceled)

43. (canceled)

44. (canceled)

45. (canceled)

46. (canceled)

47. (canceled)

48. (canceled)

49. (canceled)

50. (canceled)
